(19) United States
(12) Patent Application Publication
(10) Pub. No.: US 2015/0005772 A1
(43) Pub. Date: Jan. 1, 2015

(54) MARKING DEVICE AND EVALUATING DEVICE FOR PATELLAR RESECTION

(71) Applicant: UTI Limited Partnership, Calgary (CA)

(72) Inventors: Carolyn Ruth Anglin, Calgary (CA); Erica Lee Rex, Calgary (CA); Barry Dean Wylant, Calgary (CA); John Gunnar Person, Calgary (CA); Karen Cherk Ting Ho, Calgary (CA)

(21) Appl. No.: 14/377,164

(22) PCT Filed: Feb. 5, 2013
(86) PCT No.: PCT/CA2013/000106

§ 371 Date: Aug. 6, 2014

Related U.S. Application Data
(60) Provisional application No. 61/602,918, filed on Feb. 24, 2012.
(60) Provisional application No. 61/595,279, filed on Feb. 6, 2012.

(51) Int. Cl. A61B 17/17; A61B 17/16

(52) U.S. Cl. CPC A61B 17/1767; A61B 17/1677
USPC 606/79; 606/102; 434/262

(57) ABSTRACT

A device for marking bone such as a patella is anchored to one surface of the bone, whereby the contact with the surface defines the desired resection plane, and then a cautery or other marking device is used to mark the bone relative to this plane. The marking device is removed and then the bone may be resected using techniques known in the art. A device for measuring the remaining bone allows a user to measure bone thickness and visually estimate superoinferior angle or mediolateral angle of the resected surface relative to the desired resection plane. The measuring device may optionally be used with a computer-assisted surgical system for numerical measurements.

### CROSS-REFERENCE

The present application is related to U.S. Provisional Patent Application No. 61/595,279 filed on Feb. 6, 2012 and U.S. Provisional Patent Application No. 61/602,918 filed on Feb. 24, 2012; the entire contents of which are incorporated herein by reference.

### FIELD OF THE INVENTION

The present disclosure relates to the field of orthopaedic surgical devices and, in particular, to methods and apparatus for patellar resection during total knee arthroplasty.

### BACKGROUND OF THE INVENTION

In total knee arthroplasty (TKA), worn and damaged surfaces of the knee are resected and replaced with prosthetic components. This condition is seen in patients with severe arthritis. In many cases, the patella (kneecap) is resected and this involves removal of a posterior surface of the patella using a bone saw or reamer to obtain a flat surface. A desirable cut results in a resected surface that may be substantially parallel with the anterior surface of the patella, or at another desired angle relative to the anterior surface. A low-friction polymer prosthesis is then attached to the flat surface.

Patellar resection is challenging due to the small, hard bone, indistinct landmarks, and variable geometry. An incorrect patellar cut, whether tilted or too thick or too thin, occurs in at least 10% of patellar resections, even amongst expert surgeons, which can result in clinical complications such as reduced range of motion, anterior knee pain, impingement with the femoral component, maltracking of the patella or patellar fracture.

Current techniques for resecting the patella include freehand cutting with a saw, using a sawguide, and also using a reamer to mill the bone down. U.S. Pat. No. 5,147,365 describes a pliar-like patellar osteotomy guide that comprises integral saw capture slots to guide bone resection. The pliar-like design allows a surgeon to grasp a patient's patella to facilitate resection, however, correct positioning of the device to enable accurate resection relies solely on the surgeon's skill and discretion.

Patellar clamps and surgical saw guides have also been described that are fixed onto, or around, the patella. For example, U.S. Pat. Nos. 5,021,055, 6,174,314 and United States Patent Publication No. 2008/0097450 describe surgical devices that clamp the patella to resist movement during resection and further guide the saw during resection. Positioning of such devices is typically eyeballed by the surgeon; as a result, the accuracy of resection is again left to the surgeon's skill and experience.

Reamers have also been described as being easier to use over a saw guide, however, alignment of the reamer can be challenging and therefore the resulting resected surface is not always oriented correctly. Moreover, reamers can easily be inadvertently tilted into an undesired position without the user being aware and may provide an incorrect depth if not used correctly. United States Patent Publication No. 2010/0160915 describes a reamer device that comprises a depth gauge in order to determine the depth of the cut, however, positioning of the device and accuracy of the resection is again left to the surgeon's skill and experience which can lead to asymmetry and incorrect thickness of the resection.

While these techniques can provide good clinical results some of the time, they are not without challenges. For example, freehand sawing relies on the experience and skill of the surgeon, and obtaining an accurate cut is difficult. Use of a sawguide also relies heavily on the surgeon's experience to align the sawguide, and it may be difficult to clamp the sawguide securely around the patella. For these reasons, these procedures particularly present significant challenges to surgeons having relatively limited experience with the surgical procedure, for example residents learning the technique or low-volume surgeons.

Other devices, systems or techniques have been described for improving the accuracy of patellar resection, for example computer-assisted surgery (CAS), however, these devices often require additional time and equipment and are typically invasive.

Existing instruments and methods for measuring a resected patella and verifying the accuracy of the cut are also known and utilized in an attempt to improve the accuracy of patellar resection. For example, surgical techniques that rely on the surgeon feeling the resection with the thumb and forefinger for symmetry are typically relied on, however, these techniques lack consistency and accuracy. As well, vernier-type calipers are routinely used to check the thickness of the resected patella superiorly, centrally and inferiorly, however, this technique requires several measurements to be made and cannot determine mediolateral symmetry.

For at least these reasons, accurate patellar resectioning continues to be challenging and a need remains for devices and methods for guiding and for evaluating patellar resection.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present disclosure generally relates to medical devices and methods. More particularly, the present disclosure relates to devices and methods for guiding tissue resection. In exemplary embodiments, the methods and devices relate to marking and resecting tissue, and to evaluating the remaining tissue after resection. In exemplary embodiments, the methods and devices are used to mark, resect, and evaluate the resection of a patella. One of skill in the art will appreciate that these are exemplary uses only and the embodiments described herein may be used in other applications, and in particular may be used in other parts of the body or for training purposes.

In a first aspect of the present disclosure, a method for resecting tissue comprises positioning a marking device into a desired position relative to a surface of a bone, and coupling the marking device to the surface of the bone in the desired position. The method also comprises actuating the marking device thereby marking the bone to indicate a desired resection plane, uncoupling the marking device from the surface of the bone, and resecting a portion of the bone adjacent the marking.

In some embodiments, the desired position may comprise a contact plane defined by contact between the marking device and an anterior surface of a patella. The contact plane may define a resection plane that is substantially parallel therewith, and the resection plane may be substantially similar to a resection plane in which a surgeon would otherwise resect the tissue based on his/her intuition and experience. The desired position may also be a center of the patella. The surface of the bone may be premarked with a reference point or mark, and positioning the device may comprise aligning the marking device with the reference. Positioning the marking device may comprise aligning a view hole or through hole on the marking device with a reference point on the surface of the bone. The reference point may comprise the center point of the patella, and the center point may be defined as the midpoint between superior and inferior extents of the patella, and the midpoint may also be defined as the midpoint between medial and lateral extents of the patella.

In accordance with embodiments of the present disclosure, coupling the marking device may comprise holding the device against the bone with a surgeon's hand, or a clamp may be used. Coupling the marking device may comprise engaging an anchoring element against the bone, and the anchoring element may comprise a plurality of pegs. The plurality of pegs may be arranged in a polygonal shape such as a triangle. The triangle may be equilateral or non-equilateral. Certain embodiments comprise three pegs arranged in a triangle, such as an equilateral triangle. The equilateral triangle may have sides having a length of about 16 mm each. Two of the pegs in the equilateral triangle may be oriented superiorly and one of the pegs in the equilateral triangle may be oriented inferiorly.

In embodiments of the disclosure, the bone may comprise a patella, and the resection plane may be substantially parallel with an anterior portion of the patella, or the resection plane may be disposed at a fixed angle relative to the anterior surface of the patella. Actuating the marking device may comprise rotating the marking device relative to the surface of the bone. The marking device may be coupled to the bone along an axis that is orthogonal to a surface of the bone, and the marking device may be rotated in a plane substantially orthogonal to the coupling axis. Marking the bone may also comprise marking a line on the bone. The line may extend at least 180 degrees around the bone. The marking device may comprise an electrosurgical device such as a cautery device or other marking instrument such as a marker pen may be used to mark the bone.

According to some embodiments of the disclosure, uncoupling the marking device may comprise disengaging an anchoring element from the bone. Uncoupling the marking device may also comprise releasing a thumb and finger from the marking device.

In some embodiments, resecting the bone may comprise sawing the bone along the marking, adjacent to it, parallel to it, under the marking or on top of the marking, and the resection plane may be generally parallel to the surface of the bone. In a particular embodiment, resecting the bone may comprise sawing or reaming the bone along the marking, and the resection plane may be generally parallel to the surface of the bone. The bone may comprise a patella, and the surface may be an anterior surface of the patella.

In some embodiments, the marking device may comprise an anchoring element for anchoring the device and also a marking instrument for marking the tissue and the method may further comprise adjusting the distance between the anchoring element and the marking instrument. The method may further comprise orienting the device relative to the bone so that a portion of the device faces superiorly. In alternative embodiments, the device may be oriented in other known directions such as inferiorly, medially or laterally.

In some embodiments, the natural thickness of the bone may be measured prior to resection thereof. The marking device may then be adjusted so that it marks the bone at a distance from the anchoring surface that is calculated based on the natural bone thickness less the thickness of the required amount of bone to be removed.

In another aspect of the present disclosure, a marking device for use in tissue resection comprises an elongate central member, an anchoring arm, an anchoring element, and a marking arm. The anchoring arm has a first end and a second opposite the first end. The second end is coupled with the central elongate member. The anchoring element is coupled to the first end of the anchoring arm. The marking arm is coupled to the central elongate member, and the marking arm comprises a marking instrument coupled thereto. The marking instrument may be adapted to mark tissue and it may move relative to the anchoring element.

In some embodiments of the present disclosure, the elongate central member may comprise indicia for measuring distance, such as the thickness of the bone tissue before or after resection. Also, the device may further comprise a locking mechanism coupled with the elongate central member and the anchoring arm. The locking mechanism may be adapted to prevent movement therebetween. For example, in some embodiments, the locking mechanism may comprise a setscrew.

According to some embodiments, the anchoring arm may be adjustably movable relative to the elongate central member, and the anchoring arm may slidably engage the elongate central member.

In further embodiments, the anchoring element may comprise a base or plate having a plurality of pegs that are disposed therein, and the pegs may be adapted to anchor the device against the tissue. The device may be supplied as a kit with differing sizes of bases or plates that accommodate various sizes of bones. The anchoring arm may be rotatably coupled with the base or plate. The plurality of pegs may comprise three pegs arranged in a triangle, and in certain embodiments the triangle is an equilateral triangle having sides with a length of about 16 mm. Two of the pegs in the equilateral triangle may be oriented superiorly and the remaining peg may be oriented inferiorly. The base or plate in the anchoring element may comprise indicia for indicating a superior or inferior orientation of the device relative to a patient. The pegs may comprise conical distal tips adapted to engage bone. The anchoring element may comprise a through hole or view hole disposed therein, and that is adapted to allow visualization of a target therethrough. A crosshair may be disposed in the through hole, and the crosshair may be adapted to facilitate centering of the device over the target. In alternative embodiments, a dot, crosshair or other mark may be made on the target tissue and then the device may be centered thereover by observing the mark through the through hole.

In some embodiments, the marking arm may comprise a collar or ring for engaging a marking instrument. The marking instrument may comprise an electrosurgical instrument or a marking pen. The marking arm may be movable relative to the anchoring arm. The tissue to be resected may comprise bone, and the anchoring element anchors to the bone along an axis. In the case of patellar resection, this axis may be perpendicular to the anterior surface of the patella. The marking arm may rotate in a plane substantially orthogonal to the axis. The marking arm may have a longitudinal axis, and the marking instrument may rotate in a plane substantially orthogonal to the longitudinal axis of the marking arm.

In still another aspect of the present invention, a method for evaluating remaining tissue after resection comprises positioning a measuring device into a desired position relative to a first surface of a bone after resection thereof. The measuring device comprises a first base or plate and a second base or plate. The method also comprises coupling the first base or plate to the first surface of the bone in the desired position, engaging the second base or plate with a resected surface of the bone, and measuring thickness of the bone. Also, the method comprises measuring an angle of the resected surface relative to the first surface of the bone.

In some embodiments of the disclosure, the desired position may comprise a center of the bone, and the bone may comprise a patella. The center of the bone may comprise a midpoint of the medial and lateral extents of the bone, and also the center of the bone may comprise a midpoint of the superior and inferior extents of the bone. The desired position may be any other position including those disclosed elsewhere in this specification.

According to some embodiments, the first base or plate may comprise a plurality of pegs, and coupling the first base or plate to the first surface may comprise engaging the plurality of pegs with the first surface. The plurality of pegs may be disposed around the center of the bone. Coupling the first base or plate may comprise holding the first base or plate against the bone with a surgeon's fingers and thumb, or with a clamp. Coupling the first base or plate may comprise observing a marked portion of the bone through an alignment hole or view hole disposed in the first base or plate. Observing the marked portion may comprise aligning the marked portion of the bone with the alignment hole or with a crosshair disposed in the alignment hole.

In some embodiments, engaging the second base or plate with the resected surface may comprise advancing the second base or plate toward the first base or plate until a surface of the second base or plate abuts the resected surface. The second base or plate may be substantially flush with the resected surface.

In further embodiments, measuring the thickness may comprise reading a scale disposed on the measuring device. The scale may indicate thickness of the remaining bone after resection, and the scale may be proportional to the distance between the two bases or plates.

According to embodiments of the disclosure, the measured angle may comprise a superoinferior angle or mediolateral angle of the resected surface relative to the first surface. The measuring device may comprise a frame and the second base or plate may be pivotably coupled thereto. Measuring the angle may comprise observing an angle between a surface of the base or plate and the frame. In other embodiments, measuring the angle may also comprise measuring a first angle and then measuring a second angle different than the first angle. The measuring device may include a scale or index for quantifying or otherwise facilitating measurement of the first or second angles.

In other embodiments of the present disclosure, the bone may comprise a patella, and the first surface may comprise an anterior surface of the patella. The method may further comprise conducting additional resection of the bone based on the measured thickness or angle.

In further embodiments, the device may further comprise a marker array adapted for use with a computer-assisted surgery system. Measuring the thickness and/or angle may comprise detecting position and orientation of the marker array with the computer-assisted surgery system, and also digitizing divot positions on the measuring device.

In yet another aspect of the present invention, a device for evaluating remaining tissue after resection comprises an elongate central member, an anchoring arm, an anchoring element, a measuring arm and a swivel base or plate. The anchoring arm has a first end and a second end opposite the first end, and the second end is coupled with the central elongate member. The anchoring element is coupled to the first end of the anchoring arm, and the anchoring element is adapted to engage a first surface of a bone. The measuring arm has a first end and a second end opposite the first end, and the second end of the measuring arm is coupled with the central elongate member. The swivel base or plate is pivotably coupled to the first end of the measuring arm, and the swivel base or plate is movable relative to the anchoring element. The swivel base or plate is adapted to engage a resected surface of the bone substantially flush thereto.

In certain embodiments of the disclosure, a gap between the swivel base or plate and the measuring arm may provide a visual indicator of a superoinferior angle or a mediolateral angle of the resected surface of bone relative to the first surface. The device may also comprise a scale that is operably coupled therewith and the scale may indicate a superoinferior angle or a mediolateral angle of the resected surface of the bone relative to the first surface.

In some embodiments, the device may be supplied in a kit having several sizes of swivel bases or plates in order to accommodate various sizes of bone.

According to some embodiments, the elongate central member may comprise indicia for measuring thickness of the remaining bone after resection. The measured thickness may be proportional to distance between the anchoring element and the swivel base or plate.

In further embodiments, the anchoring arm may be adjustably movable relative to the elongate central member. The anchoring arm may slidably engage the elongate central member.

In accordance with some embodiments, the anchoring element may comprise a base or plate having a plurality of pegs disposed therein. The pegs may be adapted to anchor the device against the tissue. The pegs may have conical distal tips that are adapted to engage bone and therefore facilitate with anchoring the device with bone. The plurality of pegs may comprise three pegs arranged in a triangle, and in some embodiments, an equilateral triangle. The equilateral triangle may have sides having a length of about 16 mm. Two pegs in the equilateral triangle may be oriented superiorly and one of the pegs in the equilateral triangle may be oriented inferiorly.

In further embodiments, the anchoring element may comprise a base or plate, and the base or plate may comprise indicia for indicating a superior or inferior orientation of the device relative to a patient. The anchoring element may also comprise a through hole or view hole disposed therein, the hole being adapted to allow visualization of a target therethrough. The device may also comprise a crosshair disposed in the through hole, and that is adapted to facilitate centering of the device over the target tissue or a reference mark on the target tissue. The device may also be provided as a kit with several anchor element bases or plates that accommodate various sizes of bone.

According to some embodiments, the measuring arm may be movable relative to the anchoring arm. It may be slidably engaged with the elongate central member. In further embodiments, the swivel base or plate may be pivotably coupled to the measuring arm with a ball joint.

In still another aspect of the present disclosure, a computer assisted surgical system comprises one or more marker arrays coupled with the measuring device described in the present disclosure. The system also comprises a computer-assisted surgical system. The computer-assisted surgical system is adapted to detect the one or more marker arrays and determine angle of the resected surface and depth of the remaining bone relative to a surface of the bone. In some embodiments, this information may be digitized, processed and stored as required. In further embodiments, the computer-assisted surgical system may be adapted to optically detect the one or more optical marker arrays.

In another aspect of the present disclosure, a method for training a surgeon to perform patellar resection comprises coupling a marking device to a patella. The patella may be a patient's patella or a model of a patella. The method also comprises marking the patella with the marking device to indicate a region for resection, and removing the marking device from the patella. The surgeon then compares an intended resection plane with the marked region before resecting the patella or the surgeon compares the patella after resection with the marked region. In some embodiments of the disclosure, the method may further comprise coupling any measuring instrument to the resected patella and determining thickness thereof and/or a resection angle of the resected surface relative to an anterior surface of the patella.

### BRIEF DESCRIPTION OF THE DRAWINGS

These and other features of the invention will become more apparent in the following detailed description in which reference is made to the appended drawings.

FIG. 1 illustrates basic anatomy of a knee joint;

FIG. 2A illustrates a perspective view of a resected patella;

FIG. 2B illustrates a side view of the resected patella shown in FIG. 2A;

FIG. 3 illustrates coupling of a patellar prosthesis with the resected patella shown in FIGS. 2A and 2B;

FIG. 4A illustrates a side view of an exemplary embodiment of a marking device, according to embodiments of the present disclosure.

FIG. 4B illustrates a partial exploded view of the marking device shown in FIG. 4A, according to embodiments of the present disclosure;

FIG. 4C illustrates a top view of a portion of the marking device shown in FIG. 4A, according to embodiments of the present disclosure;

FIG. 4D illustrates a bottom view of a portion of the marking device shown in FIG. 4A, according to embodiments of the present disclosure;

FIGS. 5A-5C illustrate an exemplary method of using the device shown in FIG. 4A, according to embodiments of the present disclosure;

FIG. 6A illustrates a side view of an exemplary embodiment of a device for evaluating tissue after resection, according to embodiments of the present disclosure;

FIG. 6B illustrates a close-up side view of the device for evaluating tissue after resection shown in FIG. 6A, according to embodiments of the present disclosure;

FIG. 6C illustrates a top view of a portion of the device for evaluating tissue after resection shown in FIG. 6A, according to embodiments of the present disclosure;

FIG. 6D illustrates a bottom view of a portion of the device for evaluating tissue after resection shown in FIG. 6A, according to embodiments of the present disclosure;

FIGS. 7A-7B illustrate an exemplary method of using the measuring device shown in FIGS. 6A-6D, according to embodiments of the present disclosure;

FIG. 8 illustrates an exemplary embodiment of a measuring device that may be used with a computer-assisted surgical system; according to embodiments of the present disclosure;

FIG. 9 illustrates another exemplary embodiment of a measuring device that may be used with a computer-assisted surgical system, according to embodiments of the present disclosure;

FIG. 10A is a pseudo-radiograph created from a CT scan providing an axial (skyline) view of a left knee;

FIG. 10B is a pseudo-radiograph created from a CT scan providing a sagittal (lateral) view of a left knee;

FIG. 11A is a surface model representation of surgeon-derived resections of the patellae with the most and least consistently drawn axial resection lines;

FIG. 11B is a surface model representation of surgeon-derived resections of the patellae with the most and least consistently drawn sagittal resection lines; and

FIG. 12 is an illustration of the final peg configuration used to align the resection to the desired resection plane.

### DETAILED DESCRIPTION OF THE INVENTION

### Definitions

As used herein, the term “about” refers to an approximately +/−10% variation from a given value. It is to be understood that such a variation is always included in any given value provided herein, whether or not it is specifically referred to.

Unless defined otherwise, all technical and scientific terms used herein have the same meaning as commonly understood by one of ordinary skill in the art to which this invention belongs.

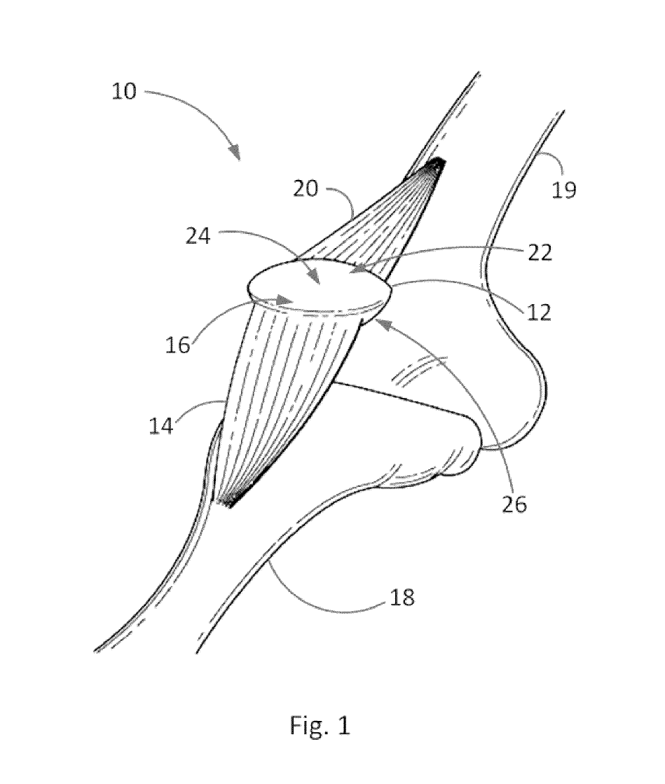

Referring to FIG. 1, there is shown the basic anatomy of the knee joint 10. A knee joint 10 is a large and complex joint that comprises the femur 19, tibia 18, and patella 12. The patella 12 is a small disk-like bone which protects the joint and also helps to increase the moment arm of the quadriceps muscle. The patella 12 glides along the surface of the femur 19 as the knee is flexed and extended. The fibula (not illustrated) is also considered a part of the knee joint, but has negligible movement. FIG. 1 illustrates the patella 12 in a non-everted position. Patellar tendon 14 attaches a distal end 16 of the patella to the tibia 18. Quadriceps tendon 20 attaches a proximal end 22 of the patella 12 to femur 19. Patella 12 has an anterior surface 24 and a posterior articular surface 26. The posterior articular surface 26 has a layer of cartilage which normally allows for smooth and painless movement. Over time, portions of the knee joint can wear out, and diseases such as osteoarthritis or rheumatoid arthritis can result in damage to the joint. Total knee arthroplasty is the current recommended solution for patients suffering from severe cases of arthritis as a means of reducing pain and restoring joint function. In the procedure, damaged surfaces of the knee joint are replaced with prostheses. If healthy, the patella may be left intact. In other cases, the patella may be resected.

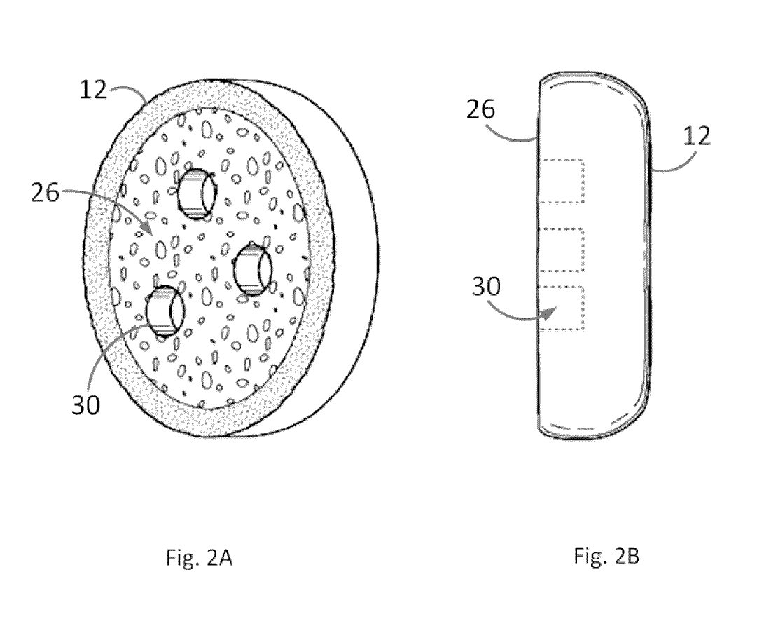

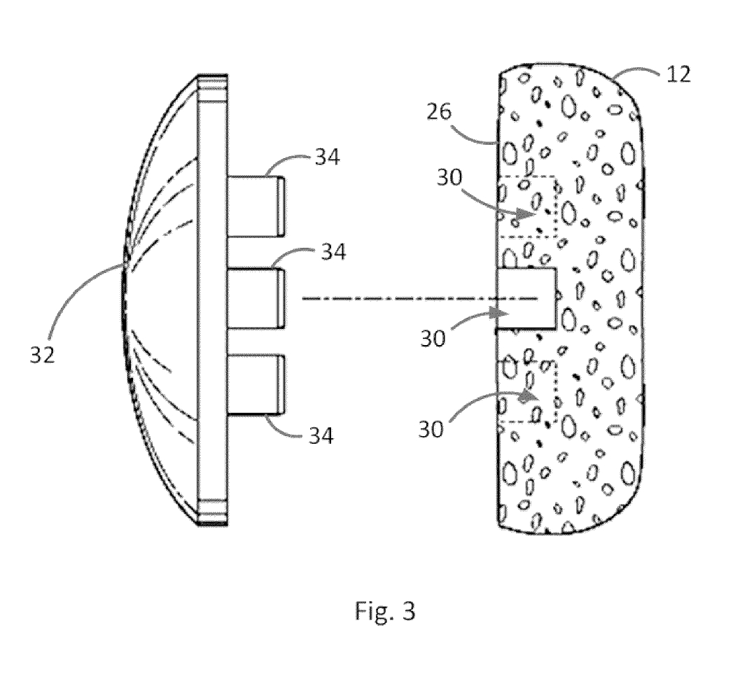

FIG. 2A illustrates a posterior surface 26 of the patella 12 after resection. FIG. 2B illustrates a sideview of the resected patella. In total knee arthroplasty, typically three engagement holes 30 may also be drilled, or otherwise formed in the posterior surface for engagement with a patellar prosthesis. FIG. 3 illustrates engagement of a patellar prosthesis 32 with the posterior surface 26 of the patella 12. In this example, three cooperating engagement pegs 34 on the patellar prosthesis are inserted into the corresponding engagement holes 30 in the patella in order to help align and couple the two pieces together. The pegs may then be secured to the corresponding engagement holes using bone cement or by other means known in the art.

As illustrated in FIGS. 1 to 3, the symmetry and thickness of the resection is determinative to the success of total knee arthroplasty. A symmetric cut is determined in reference to the anterior surface of the patella, for example, parallel to the anterior surface of the patella or at a fixed angle.

The present disclosure relates to devices and methods for guiding tissue resection without constraining the surgeon to a particular cut, often found with most devices currently being used. According to embodiments of the disclosure, the devices and methods allow the user to define the desired resection plane in a fast and simple manner.

The resection plane is marked directly onto the surface of the bone to provide a guide for the resection prior to actual resectioning of the bone. In this way, the devices and methods according to embodiments of the present disclosure offer the user the flexibility to confirm the desirability of the resection plane, and make any corrections to the resection, before resecting the bone. The user can then use their resectioning technique of choice for the resectioning procedure. Accordingly, the user can continue to use the surgical technique that they are most comfortable with for the resectioning of the bone with greater confidence and accuracy. This ability to check the marked resection plane prior to actually performing the resection offers surgeon's an opportunity to review and improve their technique. In this way, the devices and methods according to embodiments of the present disclosure are amenable for use by inexperienced surgeons, for example residents learning the technique or low-volume surgeons. The devices and methods according to embodiments of the present disclosure, by offering iterative feedback to the surgeon, can be used as training tools and methods.

The present disclosure further relates to devices and methods for evaluating and validating the resection made to the bone. According to embodiments of the disclosure, the devices and methods allow the user to visually check the accuracy of the resection, thereby, offering the user the opportunity to confirm and/or improve the accuracy of the resection made. In further embodiments, the devices and methods offer the user intraoperative three-dimensional feedback on the accuracy of the resection. In this way, the visual confirmation can be made quickly and efficiently during the surgical procedure.

### Marking

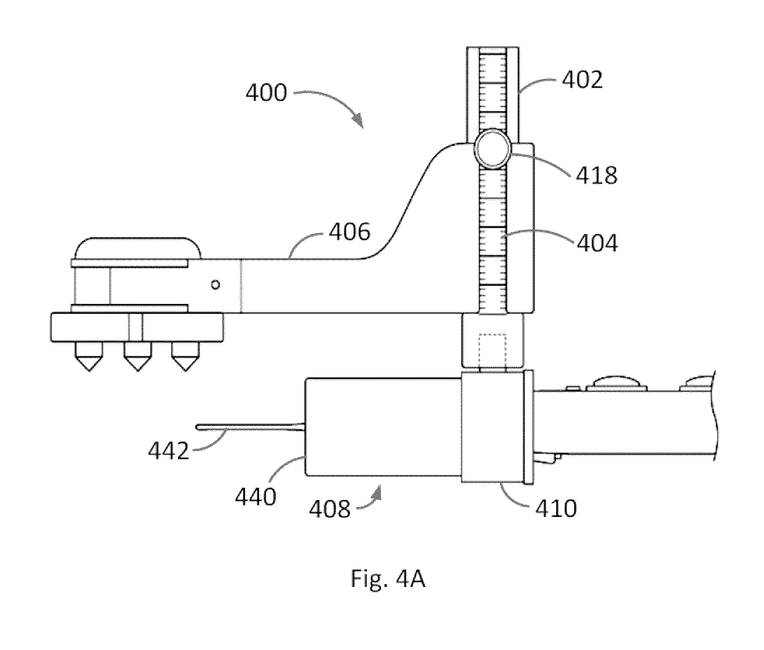

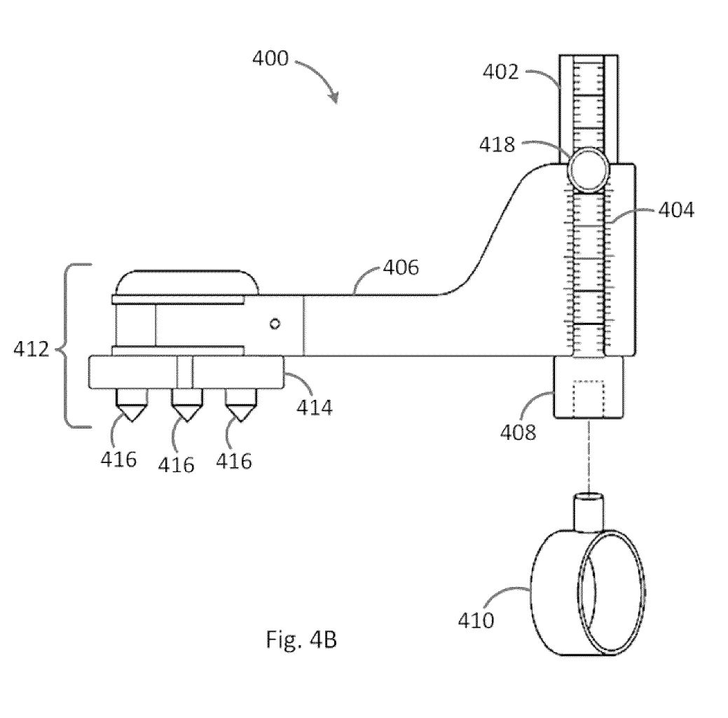

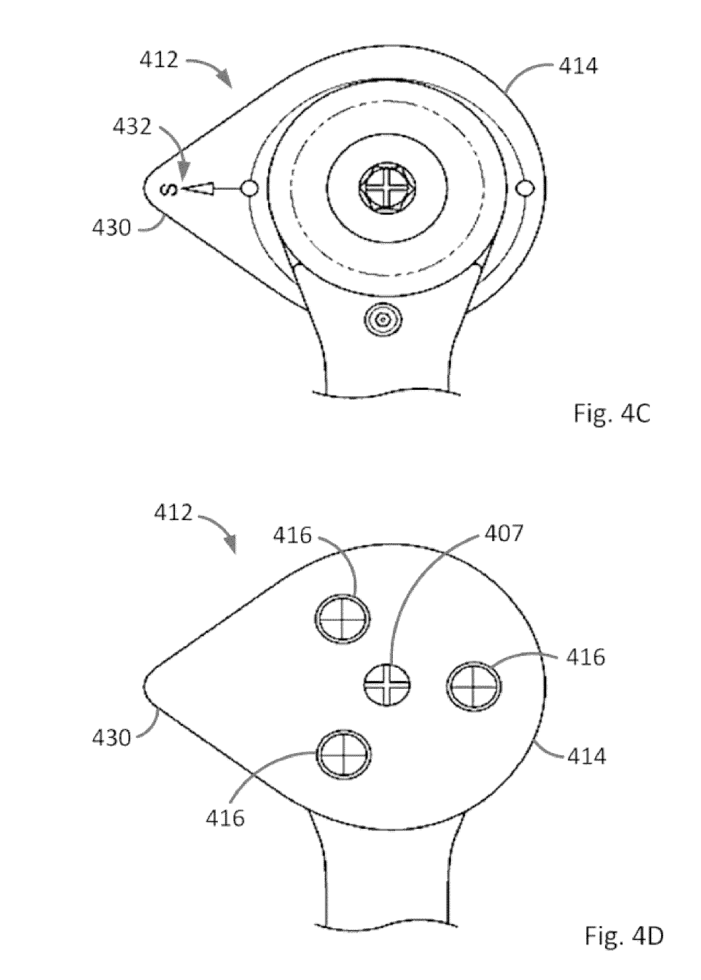

FIG. 4A illustrates an exemplary embodiment of a device 400 for marking the region of the patella to be resected. The device 400 includes an elongate central member 402 such as a strut or beam to which an anchoring arm 406 and a marking arm 408 are coupled. In some embodiments, the marking arm 408 is fixedly coupled to the elongate central member 402, however as shown in FIG. 4A, it may also be removably coupled thereto. The elongate central member 402 is slidably disposed in a channel in the anchoring arm 406. In this embodiment, the elongate central member 402 has a dovetail cross-section which slides in a matching dovetail groove in the anchoring arm 406. Thus, the elongate central member 402 may be extended and collapsed relative to the anchoring arm 406. Similarly, as the elongate central member 402 is extended and collapsed, the marking arm 408 also extends and collapses relative to the anchoring arm 406. This increases or decreases the distance between the marking instrument 442, coupled to the marking arm 408, and the anchoring arm 406, and allows the device 400 to accommodate various tissue thicknesses. Indicia 404 on the elongate central member 402 allows a user to measure a distance between the anchoring arm 406 and the marking arm 408 as the central member 402 is extended and collapsed. In some embodiments, the indicia 404 can be used to display a thickness of the tissue positioned between the anchoring arm 406 and the marking arm 408. In other embodiments, the indicia 404 can be used to display the expected thickness of the bone remnant prior to performing the resectioning procedure. A locking mechanism 418 coupled to the elongate central member 402 and the anchoring arm 406 locks the elongate central member 402 into position relative to the anchoring arm 406. In this embodiment, for example, a set screw 418 is shown.

The marking arm 408 also includes a holding fixture 410 such as a collar or ring for holding a marking instrument 442 such as a marker pen or an electrosurgical device such as a cautery device. In some embodiments, the holding fixture 410 may be adjustable to hold a variety of sizes of marking instruments 442. For example, in embodiments comprising a ring, the ring may be adjustable to hold a variety of sizes of marking instruments 442. In other embodiments, adapter sleeves 440 may be placed over the marking instrument 442 and the adapter sleeve 440 held in the ring 410. In this way, marking instruments 442 of a variety of sizes can be accommodated by the device 400.

In a preferred embodiment, the holding fixture 410 is adapted to swivel the marking instrument 442 in a plane substantially orthogonal to the longitudinal axis of the elongate central member 402. In some embodiments, the ring 410 is coupled to the marking arm 408 using a magnet as the coupler. In this way, design complexity is minimal and permits not only the desired movement, but also allows the ring 410 to be releasably coupled to the marking arm 408. Thus, a marking instrument 442 may be easily inserted into the ring 410 away from the surgical site, and then the ring 410 and marking instrument 442 may be coupled with the marking arm 408.

FIG. 4B more clearly illustrates disengagement of the ring 410 from the marking arm 408. In this exemplary embodiment, ring 410 is magnetically coupled with marking arm 408. One end of the anchoring arm 406 is slidably coupled with the elongate central member 402, and the opposite end of the anchoring arm 406 is coupled with an anchoring element 412. The anchoring element 412 comprises a contact configuration that facilitates coupling the device 400 to the surface of the bone in the desired position for defining a contact plane and ultimately the desired resection plane.

The contact configuration is established by a plurality of pegs for anchoring the device 400 against the surface of the bone. In the embodiment illustrated in FIGS. 4A and 4B, the anchoring element 412 includes a base or plate 414 having three pegs 416 coupled thereto. The pegs 416 can be arranged in any configuration, but preferably are arranged to form a polygon, more preferably to form a triangle, and even more preferably are arranged in an equilateral triangle. In preferred embodiments used on a patella, the sides of the equilateral triangle are about 10 mm to about 30 mm long, in more preferred embodiments the sides of the equilateral triangle are about 14 mm to about 18 mm long, and in even more preferred embodiments, the sides of the equilateral triangle are about 16 mm long. Other dimensions and arrangements may be used especially when using the marking device 400 with other parts of the body. Additionally, one of skill in the art will appreciate that the pegs 416 may be arranged in other configurations such as a polygon, a triangle including non-equilateral triangles, etc. However, using the preferred configuration of an equilateral triangle has been found to have a number of advantages. Some of these include minimizing the number of pegs 416 in the assembly, as well as allowing the anchoring element 412 to engage the uneven anterior surface of the patella in a stable manner. Additionally, using this configuration has been determined to provide the user with an easy way of centering the anchoring element 412 over the patella and defining the anterior surface of the patella and the resulting desired resection plane which would otherwise be difficult to estimate given the lack of anatomical landmarks on the patella. Defining the anterior surface of the patella then allows the marking device 400 to mark the patella to achieve a cut that closely approximates the resection plane that a surgeon would otherwise manually estimate based on his/her experience and training. In the embodiments shown, conical tips on the pegs 416 prevent the anchoring element 412 from inadvertently moving while engaged with the bone, without digging into the bone an appreciable distance which could compromise accurate measurement of the bone thickness.

In some embodiments, the anchoring arm 406 is pivotably coupled to the anchoring element 412. Thus, anchoring arm 406 can be rotated in a plane generally orthogonal to a central axis of the anchoring element 412 extending through the center of the pegs 416. When the anchoring element 412 is engaged with the bone, the anchoring arm 406 will then rotate relative thereto. This means that the marking arm 408 will also rotate relative to the anchoring element 412, and thus the marking instrument 442 will be able to scribe or otherwise mark the bone tissue as the anchoring arm 412 is rotated. As shown in the embodiment illustrated in FIG. 4B, as the anchoring arm 412 rotates, the ring 410 will also swivel, allowing further flexibility for the marking instrument 442 to rotate and circumscribe the bone or other tissue to be marked. Additionally, in some embodiments the marking instrument 442 may be slidably advanced or retracted in the ring 410 to help ensure that the marking instrument 442 properly engages the bone or tissue being marked.

The marking device 400 may be fabricated from metals such as stainless steel, anodized aluminum, titanium, or polymers such as Nylon, PVC, ABS, or other polymers commonly used for surgical instruments. Preferably the device 400 may be cleaned and resterilized for later use. In other embodiments, the device 400 is a single use device that is disposed of after treating one patient.

FIG. 4D illustrates an embodiment of the bottom surface of the anchoring element 412 of the marking device 400. In this embodiment, the base or plate 414 includes three pegs 416 with conical tips that help anchor the device 400 with the tissue. As discussed above, in preferred embodiments the three pegs 416 are arranged in an equilateral triangle. In this embodiment, the base or plate 414 is configured with positioning indicia for identifying the orientation of the anchoring element 412. For example, the anchoring element 412 may itself be shaped to indicate orientation. As shown, two pegs 416 of the triangle are adjacent the chevron 430 on the base or plate 414 and therefore the two pegs 416 are oriented superiorly during use, and the third peg 416 is inferior thereto. An optional through hole or view hole 407 may be disposed through the anchoring element 412 in order to allow a user to help center the anchoring element 412 against the bone or against a mark on the bone such as a dot or crosshair. The view hole 407 may also optionally include a crosshair to help center the anchoring element against a marking on the bone.

FIG. 4C illustrates the upper surface of the anchoring element 412 of the marking device 400, according to embodiments of the present disclosure. The base or plate 414 is preferably teardrop-shaped in order to provide a chevron 430 which helps with orientation of the device during placement and use. The chevron 430 includes indicia 432 such as an arrow and/or “S” marking that indicates the superior direction. This ensures that the device is consistently placed with the chevron facing superiorly toward the patient's head. Additionally, the base or plate 414 may be rotated 180 degrees so that the device may be used with both left and right knees and with a medial or lateral approach and capsulotomy. In this way, embodiments of the present disclosure offer versatility in use, accommodating a variety of patellar shapes and sizes, medial or lateral surgical approach, and right- or left-handed surgeons.

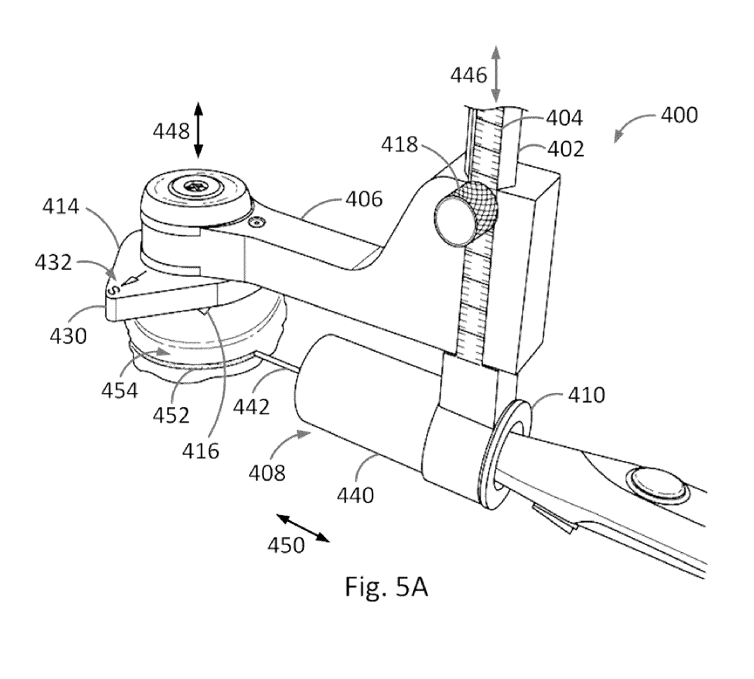

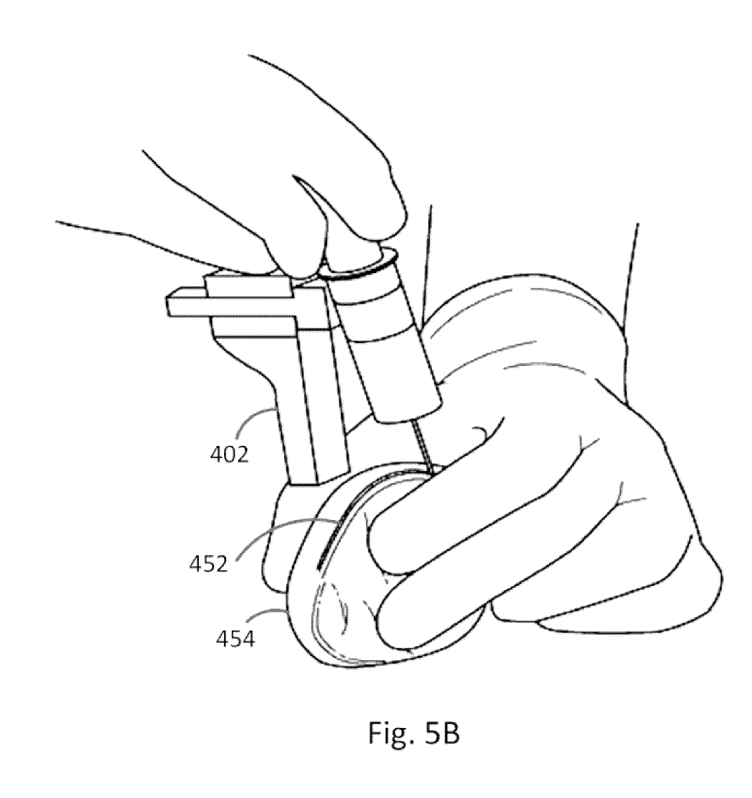

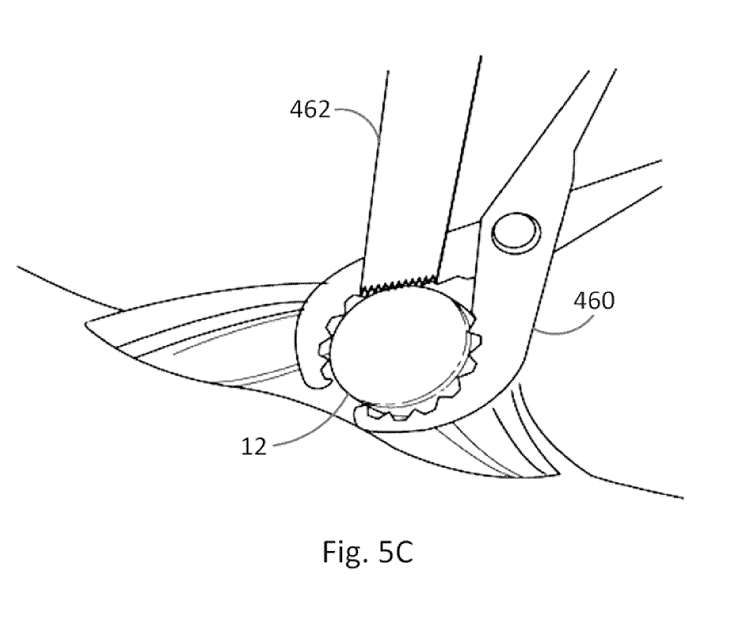

FIGS. 5A-5C illustrate operation of the marking device 400 according to embodiments of the disclosure (FIGS. 4A-4D). While use of the marking device 400 is described with respect to marking and resection of a patella, this is not intended to be limiting. One of skill in the art will appreciate that the marking device 400 may be used to mark other tissue in other regions of the body. In operation and as seen in FIG. 5A, for example, the marking device 400 is positioned adjacent the patella 454. Set screw 418 is loosened to release the central member 402 to allow slidable movement relative to the anchoring arm 406. Based on the thickness of the patellar prosthesis and the thickness of the native patella, the surgeon spreads the anchoring arm 406 and the marking arm 408 apart by sliding anchoring arm 406 along the central member 402. The anchoring arm 406 and marking arm 408 are advanced or retracted relative to one another so that the distance 443 therebetween allows the marking instrument 442 to scribe a line 452 along the patella at the desired thickness that accommodates the thickness of the patellar prosthesis. The scale 404 on the central member 402 may be used to set the distance between the arms 406, 408. Once distance has been set, set screw 418 may be tightened to lock the marking device arms 406, 408 into the desired position. A surgeon may perform this or a nurse or other operator may also perform this action away from the surgical field. Using standard surgical procedures, tissue surrounding the patella is retracted to allow access to the patella. Preferably the procedure is performed with the patella everted, but eversion may optionally be omitted if desired. The surgeon then marks the center of the patella. In some embodiments, the center of the patella can be made with a dot, crosshair or other indicia using methods and techniques known in the art. For example, the center can be estimated by feeling the extents and marking halfway therebetween. After the centerpoint is found, in some embodiments, the device 400 comprises a viewhole 407 which can be used to locate the center mark and coupling the marking device 400 to the patella.

As shown in FIGS. 5A-5C, the chevron 430 on the base or plate 414 can be rotated so that indicia 432 is facing the superior direction (toward the patient's head), in order to position the anchoring element 412 over the anterior surface of the patella engaged therewith. The pegs 416 engage the bone and provide a stable reference position for marking. In some embodiments, as shown in FIG. 5B, a surgeon may use his or her thumb and forefingers to clamp the marking device to the bone, while the surgeon's other hand is used to manipulate the marking device 400. In this way, the need for additional devices is avoided and haptic feedback is offered to the surgeon when applying the device 400 to avoid tilting the contacts off the anterior surface, for example. In other embodiments, the marking device 400 may be clamped to the patella with a surgical clamp.

The marking instrument 442 may be pre-loaded into the holding fixture which may, in some embodiments, be a ring 410 and optional adapter sleeve 440, before anchoring the marking device 400 to the patella. In other embodiments, the marking instrument 442 may be loaded into the holding fixture 410 after anchoring. The marking instrument 442, in preferred embodiments, is an electrosurgical device such as a cautery device since it allows the surgeon to mark the patella with a fine line. In other embodiments, a marker pen or other marking instrument may be used.

Once anchored into position, the marking arm 408 may be rotated around axis 448 thereby allowing the marking instrument 442 to rotate around the sides of the patella 454. Axis 448 is a vertical axis generally orthogonal to the plane in which the device 400 is anchored to the bone. In some embodiments, the marking instrument 442 may be advanced and retracted axially along axis 450 to ensure that the tip of the marking instrument 442 contacts the patella. Axis 450 is parallel to the longitudinal axis of the marking instrument 442. Additionally, in some embodiments, the ring 410 may also swivel about axis 446 in order to further ensure that the tip of the marking instrument 442 contacts the patella. Axis 446 is generally parallel to the longitudinal axis of the elongate central member 402. As the marking arm 408 is rotated, the marking instrument 442, for example a cautery device, may be activated by the surgeon, thereby creating a fine line 452 around the side of the patella at the desired thickness. The line 452 is preferably parallel to the anterior surface of the patella based on anchoring of the device 400 thereto. In other embodiments, the line 452 could be non-parallel to the anterior surface of the patella by having different lengths of pegs or an angled connection to the elongated central member. Marking may occur around a significant portion of the patella, but may not always extend 360 degrees therearound due to adjacent tissue which may be in the way. However, because the marking instrument 442 can slide in and out of ring 410, and also because the ring 410 can swivel, this allows the marking instrument 442 to access and mark a larger portion of the patella than if the marking arm 408 only rotated about axis 448. In preferred embodiments, the marker device 400 marks more than 180 degrees of the patella.

Once the patella has been marked with the desired resection line, the marking device 400 may be disengaged from the patella. The line then serves as a guide for the surgeon to resect the bone using techniques known in the art, such as by manual sawing, sawing with a sawguide, and reaming. FIG. 5C illustrates a sawguide 460 attached to the patella so that saw 462 can resect the bone. In preferred embodiments, the bone is cut slightly above the marked line so that the line remains after the sawing is completed. This allows the surgeon to evaluate the accuracy of the cut compared to the remaining marked line. The surgeon may account for this manually, or a built in offset may be included in the device so that the line is marked slightly below the actual cutting plane. Thus setting the marking distance may automatically accommodate for a surgeon who wants to cut either above or below the marked line. In still other embodiments, the bone may be resected along the marked resection line.

After resection, the surgeon evaluates the remaining bone to determine if the resection is symmetrical. In some embodiments, this may be accomplished by feeling the bone remnant with the fingers. In other embodiments, calipers may also be used to measure portions of the bone. In preferred embodiments, the symmetry of the resection is determined using the methods and devices described below.

### Measurement and Evaluation

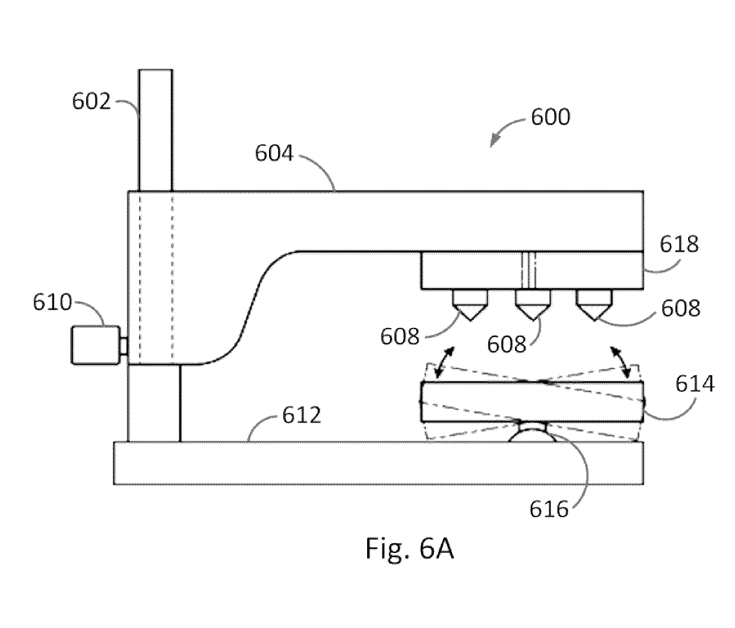

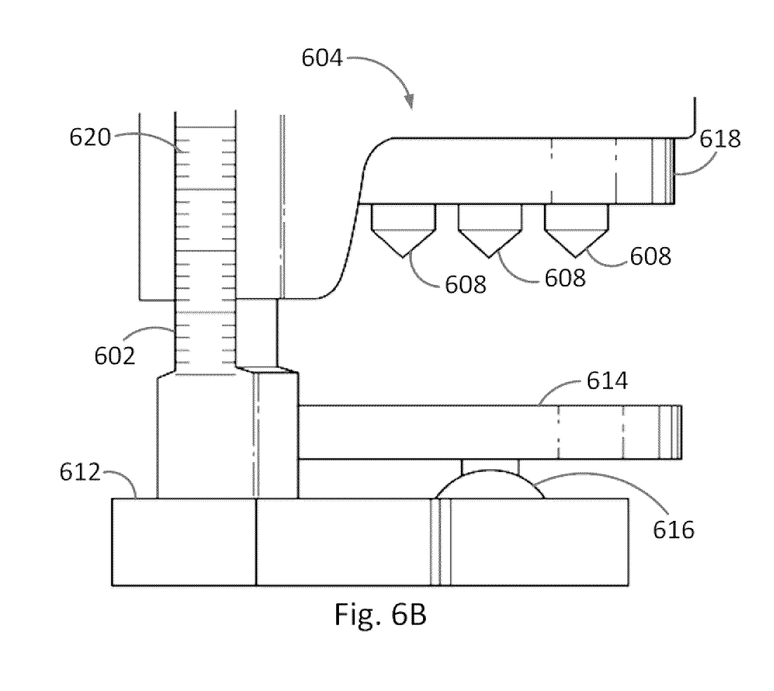

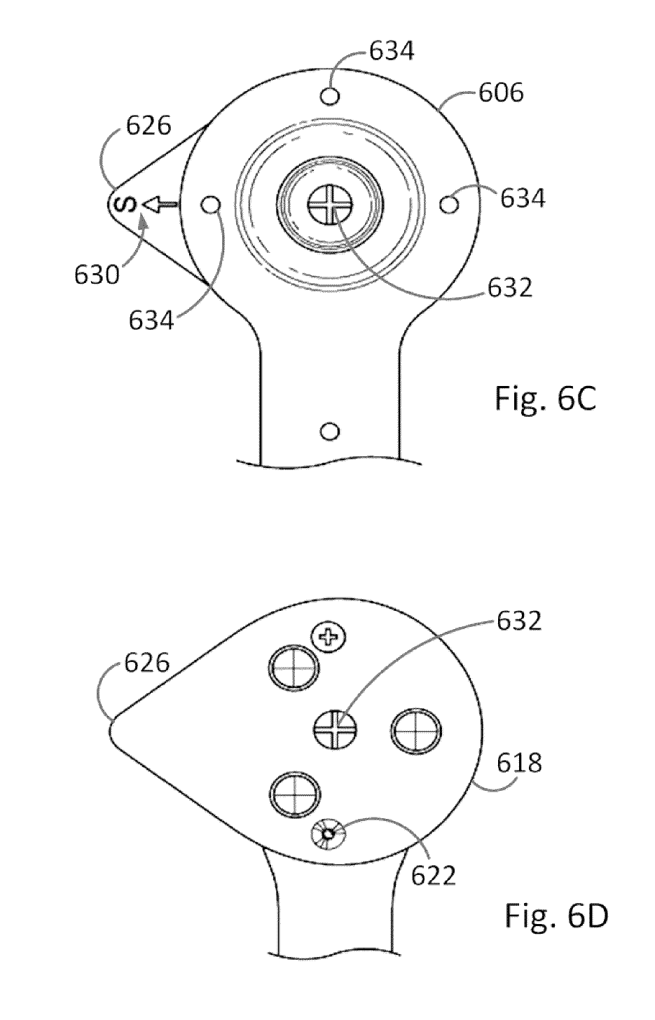

FIGS. 6A-6D illustrate an exemplary embodiment of a device 600 used to evaluate tissue after resection. The device 600 includes an elongate central member 602, for example a beam or strut, to which an anchoring arm 604 is slidably coupled. The anchoring arm 604 may be substantially similar to the anchoring arm 406 previously described above. The anchoring arm 604 includes a channel through which the elongate central member 602 may slide, such as a dovetail channel, and a locking mechanism, such as a set screw 610, may be tightened to lock the anchoring arm 604 into position relative to the elongate central member 602. An anchoring element 606 is attached to the opposite end of the anchoring arm 604. The anchoring element 606 includes a base or plate 618 having a plurality of pegs. In a preferred embodiment, as shown in FIGS. 6A-6D, the anchoring element 606 has three pegs 608. The pegs 608 are preferably configured the same as the pegs 416 in the marking device 400 described above. In some embodiments, as discussed with respect to the marking device 400, the pegs can include conical tips that help engage the tissue and prevent the anchoring element 606 from moving during use, without digging into the bone an appreciable distance to compromise accurate measurement of the thickness of the bone. Therefore, the pegs 608 are preferably arranged in an equilateral triangle, but they may be arranged in any of the configurations previously disclosed above. Based on the anatomy of the patella, the sides of the equilateral triangle preferably are about 10 mm to about 30 mm long, in more preferred embodiments the sides of the equilateral triangle are about 14 mm to about 18 mm long, and in even more preferred embodiments, the sides of the equilateral triangle are about 16 mm long. Arranging the pegs 608 in a triangle allows the anchoring element 606 to engage the uneven anterior surface of the patella in a stable manner. As discussed above, this configuration minimizes the number of components in the anchor assembly, and allows the anchoring element 606 to engage the uneven surface of the patella in a stable fashion. Also, using this configuration provides the user with an easy way of centering the anchoring element 606 over the patella and defining the anterior surface of the patella which would otherwise be difficult to estimate given the lack of anatomical landmarks on the patella. Defining the anterior surface of the patella then provides a reference from which resection can be defined and further allows the resected surface to be compared therewith to determine accuracy of the cut actually made versus what a surgeon would estimate.

A measuring arm 612 is also coupled to the elongate central member 602. The opposite end of the measuring arm 612 includes a base or plate 614 that is adapted to tilt relative to the measuring arm 612. In one embodiment, the base or plate 614 is a swivel base or plate. In preferred embodiments the swivel base or plate is coupled to the measuring arm 612 with a ball joint 616. Thus, swivel base or plate 614 can swivel in any direction relative to the measuring arm 612. FIG. 6B illustrates the scale 620 with indicia that may be included on the elongate central member 602 and the anchoring arm 604 to measure the distance between the anchoring element 606 and the swivel base or plate 614. Thus, when a resected patella is placed therebetween, the two arms 604, 612 may be advanced toward one another until firmly engaging the bone. The resulting thickness of the bone remnant may then be measured with the scale 620.

FIG. 6D illustrates a bottom surface of the anchoring element 606. The base or plate 618 is similar to the base or plate 414 previously described above in the marking device 400. As shown, the base or plate 618 is teardrop shaped with a pointed or chevron shaped tip 626. Additionally, the base or plate 618 includes a set screw 622 and pin 624 which fix the base or plate in position. The set screw 622 may be released and the base or plate 618 rotated in order to accommodate a left knee or a right knee and a medial or lateral surgical approach. An optional view hole with optional crosshair 632 is also shown in this illustrated embodiment.

FIG. 6C illustrates an upper surface of the anchoring element 606. Indicia 630 such as an arrow or “S” mark are disposed in the chevron 626 region of the anchoring element 606 and may be used to ensure that the device is oriented the right way during a procedure. Preferably, the chevron 626 points superiorly or toward the patient's head. A view hole with optional crosshair 632 is also centered in the anchoring element 606 and this allows the surgeon to visualize and place the anchoring element 606 over a pre-marked center point on the bone. Optional digitizing divots 634 may be disposed on the anchoring element 606 or other portions of the anchoring arm 604. The digitizing divots 634 may be used, in some embodiments, in conjunction with marker arrays in an optical computer assisted surgical system as will be described in greater detail below.

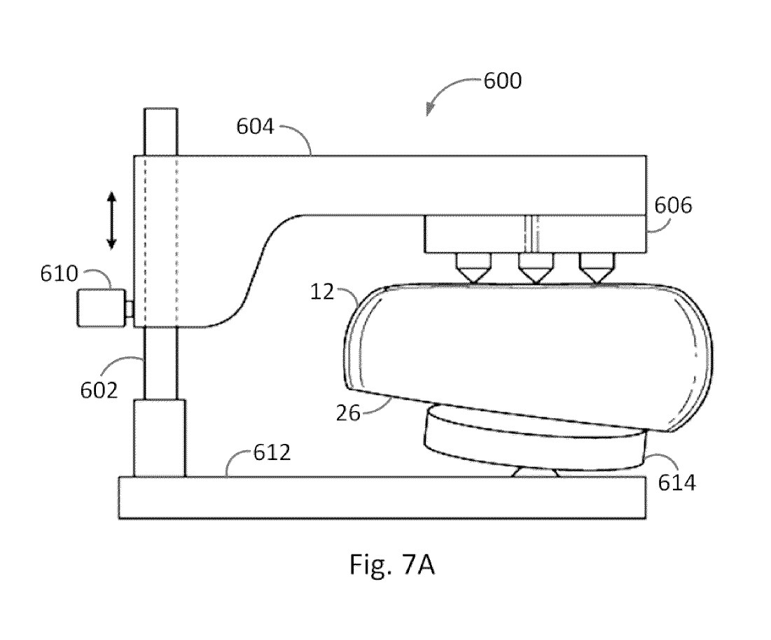

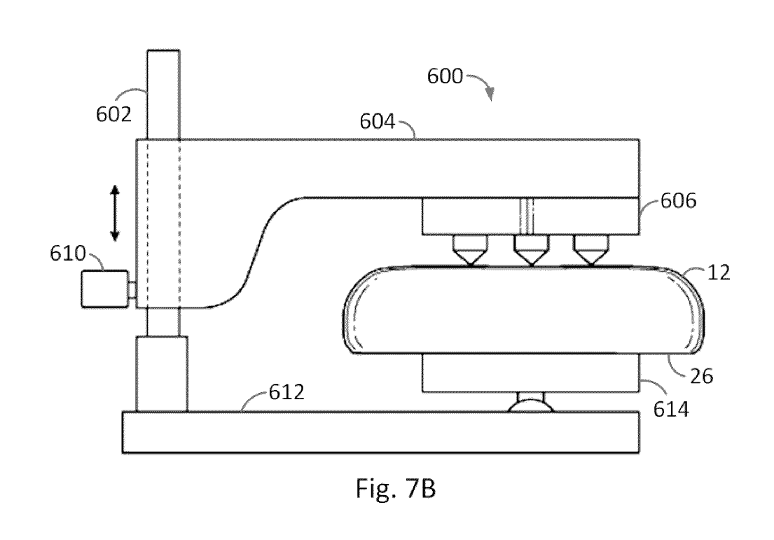

In an exemplary method of use (FIG. 7A), the measuring device 600 may be used to measure the accuracy of a patellar resection. After the patella has been marked and resected, the resected patella may then be placed into the measuring device 600. The view hole 632 may be used to help position the device over a pre-marked center point on the anterior surface of the bone. Once placed in the desired position, the anchoring element 606 is engaged with the anterior surface of the bone and the set screw 610 may be loosened. Anchoring arm 604 and measuring arm 612 are then drawn together until the anchoring element 606 and the swivel base or plate 614 engage the anterior and posterior surfaces of the bone. Once engaged, the set screw 610 may be tightened to hold the arms 604, 612 in position. The scale 620 may then be used to measure the thickness of the bone remnant. If the remaining bone is too thick, the measuring device may be removed and additional resection may be performed to reduce thickness to the desired level.

Additionally, the swivel base or plate 614 will conform to the posterior surface (the resected surface) of the patella. Because the swivel base or plate 614 is adjacent to the measuring arm 612 of the device 600 and the measuring arm 612 is also parallel to the device 600, in some embodiments, gaps between the swivel base or plate 614 and the measuring arm 612 may be used to assess the angle of the cut. Therefore, the resulting gap between the bottom of the swivel base or plate 614 and the measuring arm 612 will indicate the angle of the posterior surface relative to the anterior surface since the measuring arm 612 is generally parallel with the anterior surface of the bone.

In such embodiments, two angles will be observable. The first angle α is a mediolateral angle, and the second angle β is orthogonal thereto and represents a superoinferior angle of the resected surface relative to the anterior surface. These angles correspond to the patellar resection angles seen on the postoperative skyline and sagittal radiographs. In this way, the surgeon will have bone thickness and also be able to visually assess two angles intraoperatively to evaluate and determine accuracy and symmetry of the resection in three dimensions. If the angles are unacceptable, the measuring device 600 may be removed, and additional resection may be performed until the desired angles and thickness are achieved. FIG. 7B illustrates a preferred situation when the resected surface is substantially parallel to the anterior surface of the bone, and therefore the gap between the swivel base or plate 614 and the measuring arm 612 is similar all around which indicates that mediolateral angle α and the superoinferior angle β are also the same all around. In this way, the measuring device 600 also allows rapid visual assessment of the cut and allows quick corrections to be made. In alternative embodiments, a scale or other indicia may be included on the measuring device to provide a quantitative evaluation of any of the angles.

According to further embodiments of the present disclosure, the devices and methods for evaluating and validating the resection can be adapted for computer-assisted surgery to provide numerical feedback as well as an electronic record as an alternative to using visual feedback. In this way, the devices and methods of the present disclosure offer flexibility in design and operation.

### Computer Assisted Surgical System

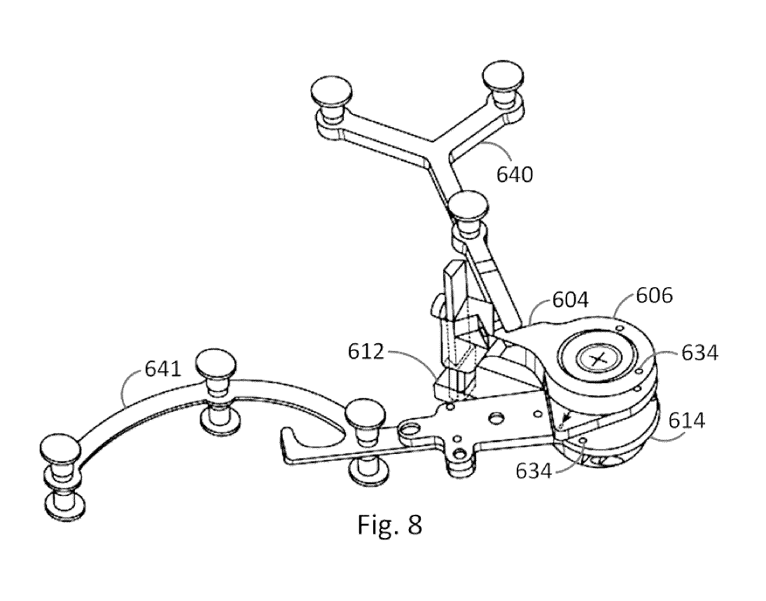

The measuring device 600 described above may be used alone, or it may also be used in combination with a computer assisted surgical system. FIG. 8 illustrates an exemplary embodiment of the measuring device 600 after it has been modified to work with a computer assisted surgical system. Y-shaped marker array 640 is coupled to the anchoring arm 604 and G-shaped marker array 641 is coupled to the measuring arm 612. The arrays 640, 641 may be fixedly coupled to the device 600, or they may be removable. Marker arrays are commercially available and known in the art. The marker arrays generally include at least three detection points so that a plane can be determined. Additionally, digitizing divots 634 may also be included on the anchoring element 606 and the swivel base or plate 614. Thus a computer assisted surgical system may use infrared or other means to detect the marker arrays and also an optoelectronic camera can be used to detect the digitizing divots 634. This allows the computer-assisted system to calculate the bone thickness and angles of the resected surface. The digitizing divots 634 illustrated in this embodiment and the following embodiment are shown on the inner or resection side of the swivel base or plate 614 for ease of visualization. However, in use, this surface may be obstructed by the patella and therefore the digitizing divots 634 may be placed on the outer surface of the swivel base or plate 614 or another adjacent surface. Similarly, for the anchoring element 606, divots 634 may be placed along any surface that is easy to measure.

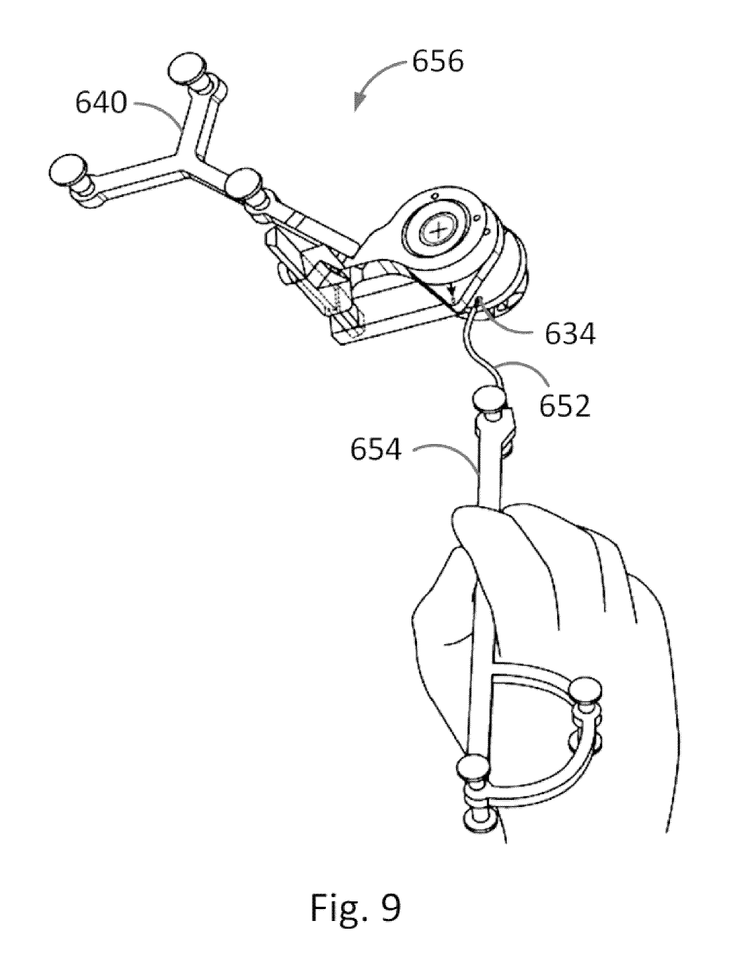

In some cases, having two marker arrays 640, 641 coupled to the measuring device 600 may make the device 600 awkward to hold and manipulate, and therefore the G-shaped marker 641 may be removed and only the Y-shaped marker array 640 is coupled with the measuring device 600. In such preferred embodiments, as shown in FIG. 9, a separate, hand-held P-shaped marker array 654 may be used with the Y-shaped marker 640 array. A surgeon or other operator may grasp the P-shaped marker 654 and manipulate it. The P-shaped marker also includes a probe 652 which can then be moved into engagement with the digitizing divots 634. In this way, by digitizing the two planes, the computer-assisted device 656, schematically illustrated in FIG. 9, allows bone thickness and angles to be calculated. The two planes can be determined by digitizing at least three points on each plane. In still other embodiments, the G-shaped marker and the Y-shaped marker may still both be used, but with an overall smaller size.

While “Y,” “G,” and “P” shaped arrays were used in these exemplary embodiments, one of skill in the art will appreciate that this is not intended to be limiting and other marker array configurations may also be used. Additionally, other non-optical computer-assisted surgical systems may be used in any of the above mentioned embodiments and therefore may not require marker arrays. For example, electromagnetic markers or accelerometer-based technologies may be used to compare the two surfaces, or any other technology that allows comparison of the angle of the two planes. Therefore, some embodiments may include the use of two marker arrays, one marker array plus digitizing; electromagnetic markers on both planes; inertial measurement units (IMUs) such as accelerometer-based units on both planes; or other electronic means of measuring the difference between two planes.

### Training

In addition to using the disclosed devices and methods for marking, resecting, and evaluating resection in a patient, they may also be used as means for training surgeons. Use of these devices generally takes the same form as described above except that instead of marking, resecting, and measuring an actual patient's patella, an artificial bone model or cadaveric specimen is used and the resulting resection is evaluated by a surgeon. In some instances, training may be conducted on an actual patient's patella. Also, the devices and methods described herein may also be used to allow a surgeon to evaluate his/her own intended resection against what the marking device recommends. This will help the surgeon improve his/her accuracy over time or can allow an expert surgeon to judge the intended cut of an orthopaedic resident.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### Defining the Patellar Resection Plane

### Contact Configuration

Fundamental to patellar resection is determining the resection plane. Although various landmark guidelines (such as medial-lateral extents) have been suggested for performing the patellar cut, the ultimate goal is to be parallel to the anterior surface, producing a visually rectangular cut, with equal thicknesses in all quadrants. Since asymmetry with respect to the anterior surface has been correlated with anterior knee pain, it is essential to reference a device off of the anterior surface.

Two surprising challenges to this are: to determine what the anterior surface plane is (although the visual goal is a ‘rectangular’ cut, the anterior surface is not flat, and is rarely considered in three dimensions), and to determine whether parallel to this is indeed what looks right to surgeons on the postoperative X-rays. Due to the ellipsoidal and variable shape of the patella, what looks obvious to one person as the anterior surface and the desired resection line is often different from what another person sees.

The purpose of this study, therefore, was (a) to determine the desired resection plane by having surgeons draw virtual resection lines on axial and sagittal preoperative X-rays; (b) to determine the corresponding estimated anterior surface plane; and (c) to determine a contact configuration on the anterior patellar surface that can achieve the desired patellar resection plane.

### Methods:

There were four stages to this study: preparing the images, obtaining surgeon input, analyzing the surgeon data, and evaluating different contact geometries to achieve the desired resection plane.

### Preparing the Images

Following ethics approval, we imaged 18 cadaveric knee specimens (9 left, 9 right; 8 male, 10 female; average age 76; range, 34-91 years) using computed tomography (CT), with a slice thickness of 0.6 mm. All of the specimens had largely normal anatomy, without obvious osteophytes, although some showed signs of early osteoarthritis.

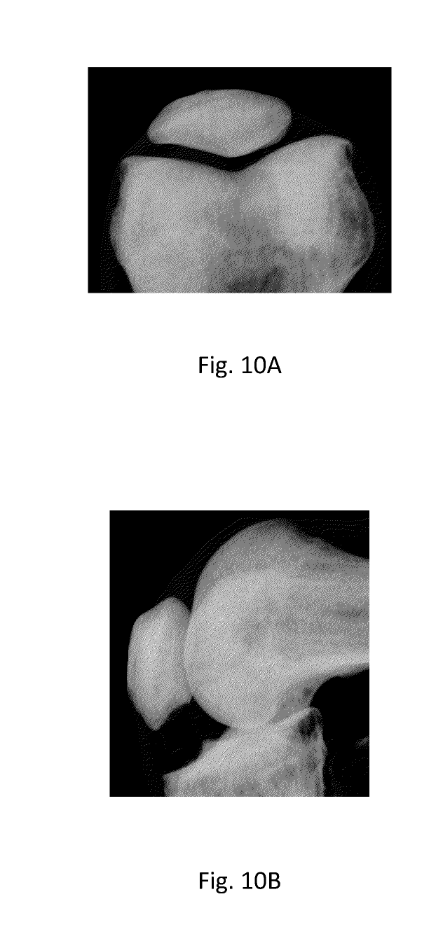

Pseudo-radiographs were created from the CT scans in the axial and sagittal directions, using Amira image analysis software (Version 5.3.1, Visage Imaging, Andover, Mass.). The CT was viewed using the volume rendering (Volren) function, which was adjusted so that the bone appeared white and the background black. The threshold was set to remove most of the soft tissue in the view, to look as similar as possible to a clinical X-ray. The image was rotated to align the knee similar to how a radiation technologist would align a patient: for the axial view, the image was aligned to provide a skyline view, where the patella is viewed from the superior looking downward (FIG. 10A); for the sagittal view, the image was rotated so that the condyles of the femur overlapped and appeared as one (FIG. 10B). Screenshots of each view were taken and the orientation saved for later analysis. Three repetitions of each of the projections were saved in randomized order, for a total of 54 axial projections and 54 sagittal projections.

### Obtaining Surgeon Input

Four experienced, fellowship-trained orthopaedic surgeons assisted in determining the desired resection plane and the corresponding anterior surface plane. Using a custom macro in ImageJ (Version 1.42q, National Institutes of Health (NIH), USA), surgeons marked the lines electronically on each X-ray; previous lines did not appear. The macro recorded the (x,y) coordinates of the endpoints of the line in a text file along with the surgeon's initials and the date.

X-ray input was performed in four passes. In the first pass, the surgeon went through the axial projections, drawing the line at which they would ideally resect the patella. In the second pass, they drew a line to represent their best estimate of the anterior surface. Third, they went through the sagittal projections, drawing their ideal resection line and the anterior surface line was 3.5° (SD=3.3°). From the visual display of the lines on the patellar circumferences, it appeared that, when asked to identify the anterior surface, the surgeons focused on a more localized portion of the anterior surface whereas the estimated resection plane approximated a symmetric resection; if the resection plane were to align with the drawn anterior surface plane, it would result in thicker medial and superior sides.

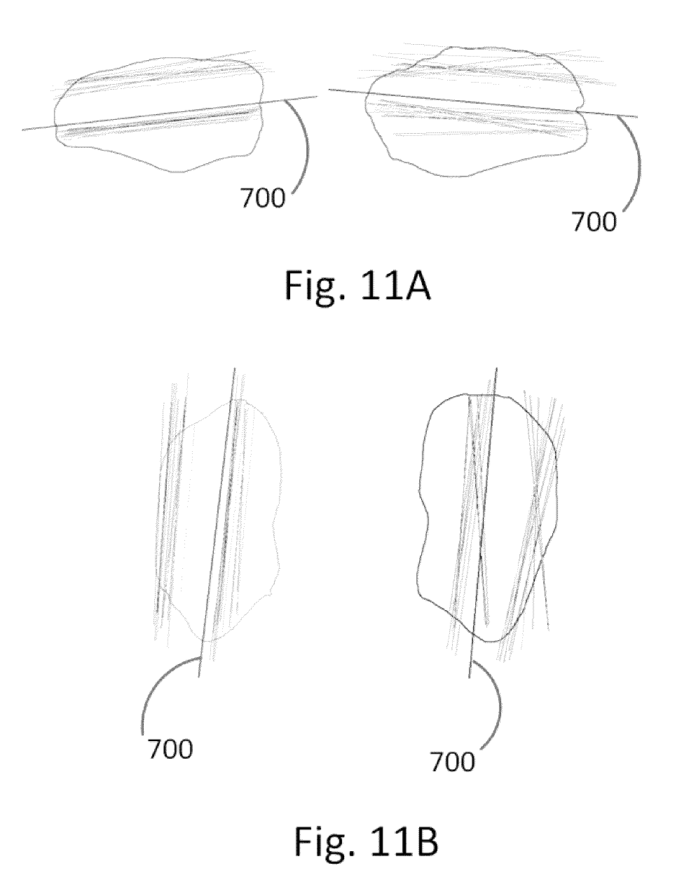

The most and least consistently drawn lines are shown on the patellar circumferences in FIG. 11A (axial) and FIG. 11B (saggital). In FIGS. 11A and 11B, the resection lines are by four different surgeons, with three repetitions each (see Graph 1 or 2 for legend). The solid black line 700 represents the corresponding peg plane (thickness is unimportant). In general, the flatter the patella, the more consistently the lines were drawn, while the more rounded or irregular, the more inconsistently they were drawn; however, this did not hold true in all cases. Also, the more well-defined the medial and lateral extents were on the axial views, the more consistently the lines were drawn, although this also did not hold true in all cases. The surgeons appeared to be taking a mental least-squares fit to the anterior surface, but varied in which part of the anterior surface they included.

### Anterior Surface Contact Configuration

The first decision in the process of defining the desired configuration was to use a system of three contact points since three points always find a stable orientation, as with a three-legged stool. With four or more points, a ring, or a flat plate, there is a risk of instability, as with a chair with one shorter leg. In general, the patellar geometry was higher in the middle and sloped off inferiorly, which explains why a ring of contacts (as with the reamer) or a flat plate can create a tilt of several degrees on some patellae. Although early investigations revealed that the flattest portion of the anterior surface was consistently in the superolateral quadrant, the pegs were too close together in this region to provide good stability; furthermore, after analysis, it was discovered that focusing the pegs in this region did not provide a symmetric resection.

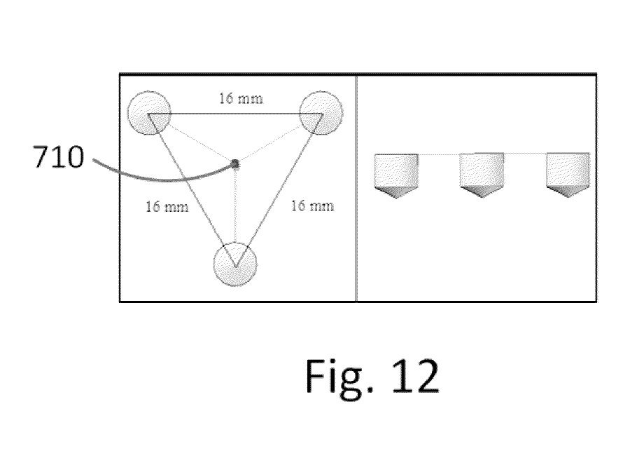

The final configuration that fit best with the surgeon-defined resection planes was a 16-mm equilateral triangle, with two points superior (medial and lateral) and one point inferior, centred on the patellar centre. The patellar centre was defined halfway between the superior and inferior extents and medial and lateral extents (FIG. 12). Referring to FIG. 12, the centre of the pegs 710 is aligned to the centre of the patella. The patellar centre is defined halfway between the superior and inferior extents and medial and lateral extents.

Furthermore, having symmetry about the centrepoint simplifies the design of the surgical instrument. The final configuration produced a better fit than having two inferior points and one superior point due to the sloping off of the surface inferiorly. We considered a 15-mm triangle for smaller patellae, but this altered the resulting angles by at most 0.3°, which was too small to be worth the practical consequences. With 16 mm sides, the distance between the inferior and superior points is 13.9 mm.

Most of the resection plane and anterior surface plane angles (53 out of 72; 15 AR, 11 SR, 14 AS and 13 SS) fell into the first category, i.e. within the surgeon averages, as expected and desired (Graph 1 and 2); 15 lines (3 AR, 4 SR, 4 AS and 4 SS) fell into the second category, i.e. within the surgeon ranges; and 4 (3 SR, 1 SS) fell into third category, outside of this range, but only by 0.96°, 0.54°, 0.40°, and 0.56°, respectively.

### DISCUSSION

On the basis of this shape analysis study, a novel peg configuration was generated for a patellar resection device that was within 1° of the range of angles that the surgeons defined as their desired resection line for all 18 patellae studied; 74% fell within the range of surgeon averages. In eight additional pairs of cadaveric knees used in our validation testing of the devices, the predicted peg results and a symmetric anterior surface were within a few degrees of each other: on average 1.7° different in the ML direction (SD=1.7°, ranging from −4.6° to +1.4°) with the predicted peg results yielding a thicker medial side (or thinner lateral side) on average and −0.2° different in the SI direction (SD=1.6°, ranging from −3.7° to +1.6°) with the predicted peg angle resulting in a thicker inferior side (or thinner superior side) on average. It is contemplated that patellar resection at an angle other than parallel to the anterior surface can be achieved by extending one or two of the pegs if desired.

The high variability in surgeon input confirms the difficulty of defining the anterior surface plane and of defining an ideal resection plane. This also highlights the difficulty of reporting patellar asymmetry or patellar tilt clinically since the definition of the patellar horizon is so variable, despite the fact that studies describe it as a fixed reference. Conversely, the high variability provides some leeway in achieving an exact resection.

The intra-surgeon and inter-surgeon repeatability were similar to a previous study with three different surgeons, in which the AR intra-surgeon repeatability using the medial-lateral extents method was 1.6° and the AR inter-surgeon repeatability was 2.0°, although the maximum differences were larger in the present study. The present study is novel in evaluating sagittal radiographs and the anterior surface, as well as in investigating the relationship between the 3D anterior surface geometry and the desired resection plane. Sagittal symmetry is important because SI asymmetry has an even stronger correlation with anterior knee pain than ML asymmetry. The same 2D-3D analysis techniques could be used for any joint in which surgeons routinely use plain X-rays.

The only quantitative definition of asymmetry that we are aware of is a difference of greater than 2 mm between the medial and lateral or superior and inferior patellar thicknesses, measured 15 mm in from the patellar extents. On an average sized patella, this results in a similar ML spacing to our peg configuration, although as previously mentioned, our 3-peg configuration produces a better fit than the 4-peg configuration that this definition may imply.

The main limitation of this study is that the surgeons' lines drawn on X-rays may not be the same plane as they would choose to resect intraoperatively. Nevertheless, it does reflect what they would like to see on the X-ray postoperatively, and is the basis for measuring asymmetry. Only one of the surgeons (Surgeon 1) routinely does preoperative planning for the patella, and was therefore most familiar with drawing lines on the X-rays (axial only); he had the smallest standard deviation and his resection lines tended to be closer to the overall surgeon average. The particular projection of the patella could also impact the resulting plane; however, all surgeons saw the same projections, so the variability within and between surgeons is unrelated to the projection. Another potential limitation is that the patellae investigated were largely healthy patellae, but since arthritis affects the posterior surface rather than the anterior surface, this should not represent a problem: a benefit of the device is that the peg configuration should produce the desired resection on a diseased patella equally well to a healthy one. The shape of the articulating patellar surface and whether it is a male or female patella, which have previously been shown to affect resection accuracy clinically, should likewise have no impact on the result since the device references solely off of the anterior surface.

Three prototype surgical devices were developed on the basis of these contact points: one to guide the resection plane, and two to evaluate the resection plane after it has been made using whatever resection technique the surgeon prefers; of the latter devices, one uses visual feedback and the other uses computer-assisted surgery. The information presented could also benefit custom rapid-prototyped surgical guides, which have been used previously for the femur and tibia, by working from similar contact points. Existing surgical devices could also be modified to incorporate the proposed contact configuration.

If a statistical shape model of the patella is available, we found that fitting a plane to the central portion of the anterior surface provides a good representation, and can be achieved automatically. This approach is useful when a 3D model of the patella is available, e.g. from a CT scan. However, since this is not available in most cases, the mechanical device with peg contacts is suitable for the real anatomical patella.

### Example 2

### Guiding Tissue Resection

### Marking Device

The purpose of this study was to evaluate the accuracy, ease of use and potential design improvements of a prototype marking device that was developed based on the contact configuration discussed above. Accuracy was judged by the mediolateral (ML) resection angle, superoinferior (SI) resection angle and difference from the intended thickness.

### Methods:

### Device Design

The central concept of the prototype device is to mark a line parallel to the anterior surface, using a cautery tool or marker pen, and then remove the device, leaving the marked line. The surgeon then uses their technique of choice (freehand, sawguide, or reamer) to align with the marked line. In this way, the surgeon can continue to use the device they are most comfortable with, but with greater confidence and accuracy. By not providing a saw slot, the device is lighter, smaller and non-invasive, and leaves the control in the surgeon's hands. The surgeon or resident can then compare the drawn line to what they would have done, learning in the process, and providing a second-thought evaluation of the patellar cut. This is similar to the common practice of drawing several guidelines on the femur (transepicondylar axis, posterior condylar axis and Whiteside's line) to see how they compare. Residents may have the opportunity to do the patellar cut earlier in their residency because the surgeon, after checking the drawn line, can feel more confident of the resulting resection.

An important aspect of the device is accurately defining the anterior surface to achieve the desired resection. The contact configuration was determined as described above and the prototype device used a peg configuration of a 16 mm equilateral triangle, with two pegs positioned superiorly and one inferiorly. The prototype design used for testing had cone-point set screws as the contact points to allow their depth to be adjusted during the initial stages of testing. The length was chosen to be short enough to promote stability while being long enough to allow visibility to apply the device to the anterior surface. The size and sharpness were tested to grab onto the bone without digging in too much.

Rotating the cautery tool around the patella is achieved using a swing arm, rare earth magnetic coupling, and a custom Delrin sleeve that fits around the cautery tool, sliding in and out of the metal collar. This sleeve could have a different inner profile for cautery tools with a different shape. The device could also be used with a marker pen, but the surgeons and residents preferred the cautery tool as it is more reliable and leaves a finer line. By pushing the cautery tool in and out of the metal collar while rotating it around the patella, the line can be drawn more than 180° around the patella, posterior to the tendon attachments, providing guidance in both the ML and SI directions.

Desired depth is set on the sliding dovetail mechanism to allow the surgeon continuous depth adjustment. It was originally intended to be set exactly at the desired depth, but through testing we discovered that it is advantageous to set the depth slightly thinner, resecting posterior to the line instead of on it, so that alignment with the marked line can be checked following resection. This could be part of the instruction procedure or could be incorporated directly into the device.

The device is held onto the patella with the thumb and forefingers, to avoid using an invasive bone screw or bulky clamping device, and to provide haptic feedback to the surgeon when applying the device to help avoid tilting the contacts off the anterior surface. Using the thumb and fingers works because the device is only used to mark the line rather than to create the saw cut, and is only held on for a short duration of time. The resulting profile provides good visibility of the patella while marking the line. The device is suitable for all patellar shapes and sizes, medial or lateral approach, with right- or left-handed surgeons.

### Artificial Bone Testing

To mimic the surgical setup in the artificial bone testing, and to perform pilot testing for design and use iterations before testing on valuable and limited cadaveric specimens, medium-sized right and left legs (Sawbones, Pacific Research Laboratories Inc, Vashon, Wash.) were set up in full extension and anchored onto a table. The patella was attached to the femur and tibia using materials simulating the tendons and lateral retinaculum, and covered with material representing skin. A standard incision represented the visibility and access during surgery.

Two custom-moulded patellar geometries were used, an approach that could be useful to other researchers, as they were more realistic than previously used commercial products and were derived from CT scans of cadaveric specimens with which we could do additional shape analyses. Geometry 1 was a left patella, smaller, regularly-shaped and considered the ‘easier’ geometry. Geometry 2 was a right patella, larger, irregularly-shaped and considered the ‘harder’ geometry. These had the most-consistently and least-consistently resection lines, respectively, drawn by the four surgeons, reflecting our best estimate of the easiest and hardest patellar geometries to resect accurately. Patellar bone models were generated from the CT scans and rapid-prototyped. A mould made from the rapid-prototyped model was used to generate the patellar bone models (Foam-it 15; SmoothOn Inc., Easton, Pa., for which the density is 15 pounds per cubic foot). The anterior surface was covered with a thin layer of Thera-band to provide compliance and to partially obscure the anterior surface. Since the foam is insulating and the cautery tool requires a conduction path, the experimenters instead dipped the cautery tool in calligraphy ink, leaving an ink line on the patella. Normal use of the cautery tool was verified during the cadaveric testing.

Two Orthopaedic Surgery residents (4^{th} and 5^{th} year) performed resections using three techniques: the prototype marking device with freehand using a surgical oscillating saw, the prototype marking device with sawguide using a standard surgical sawguide (Zimmer; Warsaw, Ind.), and using the conventional sawguide technique. For the prototype resections, the initial resection was left as is; for the conventional resections, the experimenter measured the thickness and symmetry with calipers and had the option of revising the cut until satisfied. After initial practice with the instruments and experimental setup, each experimenter performed three repetitions of each of the three techniques on the two different geometries, for a total of 18 tests each. Tests were performed in a randomized order. Procedure time was recorded, including a breakdown of the steps.

The prototype marking device procedure begins by locating the centre of the patella; this was done by feeling the height and width with the fingers, and marking the resulting centrepoint with a marker pen or cautery tool. The desired remaining thickness, determined from the patellar thickness minus the prosthesis height, was set on the depth gauge of the device and the device applied to the centre of the patella with the arrow pointing superiorly. The line was then drawn with the cautery tool more than 180° around the patella, allowing both ML and SI planes to be guided. The device was removed and the experimenter either aligned the saw or sawguide with the line to complete the cut.

The patellae were CT scanned before and after resection (0.6 mm slice thickness), followed by segmentation of the patellar bone (Amira Version 5.3.1; Visage Imaging, Andover, Mass.). The resected patellae were aligned to the original surface models using the AlignSurface function in Amira, plus manual fine tuning, and then brought into AutoCAD (Version 2010, AutoDesk, San Rafael, Calif.). In AutoCAD, an average plane was fit visually to the resected surface of the patellar model, and then the average resection plane, determined previously from the four surgeons' input on pseudo X-rays, was applied to the model. This was a particular advantage of making custom moulds of the previously-analyzed patellae. The ML and SI angles were measured between the resultant plane and the average surgeon-identified resection plane. The centre of the patella was determined from the medial, lateral, superior and inferior extents of the model, i.e. by drawing a box around the patella. The thickness from the anterior surface to the resected surface was measured at this centrepoint and then compared to the intended remaining thickness specified in the testing process (13 mm for the left, 12 mm for the right).

The angle, thickness and time data were analyzed using ANOVA, followed by Student's t-tests when significant, using PASW Statistics 17.0 analysis software (Statistical Package for Social Sciences (SPSS) Inc., Chicago, Ill.). Shapiro-Wilk tests confirmed normality of the data. Angles within ±7° were considered symmetry based on previous studies that showed greater anterior knee pain beyond this limit and represents a normal range of results.

### Cadaveric Testing

Eight pairs of fresh-frozen cadaveric knee specimens (6 female, 2 male; mean age 82, range 67 to 90 years) were used for testing, following ethics approval. They were CT scanned prior to testing and then prepared with a midline incision followed by a standard parapatellar capsulotomy: medial in 14 cases, lateral in two; this was inadvertent initially due to the truncated length, but provided the opportunity to test both approaches. Soft tissues were released to allow for eversion of the patella, and cleared around the circumference to allow for the application of the saw guide, as done clinically. The specimens varied from no arthritis to severe arthritis, with the majority having moderate arthritis (grades 2-3). The arthritic state did not affect the experiment as the devices rely on the anterior surface, not the articulating surface, one of the advantages of the device.

For each specimen pair, the prototype marking device-with-sawguide resection was performed on one side and conventional-sawguide resection was performed on the other, in randomized order. The same two residents who performed the artificial bone testing performed the cadaveric testing. The cautery tool produced a clear, precise line, about 1 mm in thickness. As with the artificial bones, in the prototype marking device case, the first cut was taken as the final cut; small corrections to the resection were allowed, such as removing a ridge but the resection plane itself was not allowed to be re-cut or otherwise modified. In the conventional case, the experimenter could correct the cut until they were satisfied; cuts after the initial sawguide cut were usually done freehand, with the patella being secured with towel clips. Experimenter 1 set the marking device such that the line would be cut off with the saw; Experimenter 2 set it such that the saw cut just below the line, leaving the line visible afterward. This latter technique had the advantage of confirming that the cut made corresponded to the cut recommended by the device. The desired thickness was determined from caliper measurements, with the prosthesis thickness being subtracted from the total thickness.

Once the resections were complete, CT images were acquired and used to calculate the desired resection plane as well as the achieved resection plane for each patella, by importing the segmented surfaces into AutoCAD. From this the ML and SI angles as well as the remaining bone thickness were measured, using the same method as for the artificial bone models. The symmetric resection plane was determined by fitting a plane to the central portion of the anterior surface, 15 mm in from the edge. The three-peg model of the device was also applied to the surface to determine the expected marking device resection angle. In other words, the former tests the symmetry relative to the surface, whereas the latter tests how closely the device achieved its expected goal. The former reflects the clinical goal whereas the latter judges the device itself. ANOVA tests of the MIL angle, SI angle, bone remnant thickness and time results were performed with p<0.05 considered significant. Normality was confirmed.

### Results:

TABLE 1
Statistical results (p-values) for validation testing

| Parameter | Experiment | Technique | Experimenter | Geometry/Side |
|---|---|---|---|---|
| ML Angle | Artificial | 0.30 | 0.67 | <0.001 |
| | Cadaveric | 0.66 | 0.29 | 0.58 |
| SI Angle | Artificial | 0.56 | 0.46 | <0.001 |
| | Cadaveric | 0.06 | 0.18 | 0.75 |
| Thickness | Artificial | 0.92 | 0.46 | <0.001 |
| | Cadaveric | <0.001 | 0.88 | 0.82 |
| Time | Artificial | 0.42 | 0.002 | 0.04 |
| | Cadaveric | 0.11 | 0.03 | 0.33 |

### ML Resection Angle

All of the ML angles in the artificial bone testing were within the symmetry limit of 7° (Graph 3a). The prototype marking device-with-sawguide and the marking device-with-freehand resections both obtained resections slightly closer on average to the desired resection plane than the conventional resections. Geometry 1 (more regular and smaller) was resected 3.1° closer on average to the desired ML plane compared to Geometry 2, which tended to be thicker medially (p<0.001).

In the cadaveric testing, the ML angle was significantly affected by the interaction of technique and side (Graph 3b): left patellae were resected significantly better than right patellae when using the conventional sawguide (p=0.047) whereas there was no difference with the prototype marking device resections. The worst symmetry, in both the conventional and the prototype cases, was for the lateral approach. The predicted peg results and the symmetric anterior surface were within a few degrees of each other (2.4° on average), confirming that the pegs predicted a symmetric resection.

### SI Resection Angle

In the artificial bone testing, all but one resection (with the conventional saw guide) were within the symmetry limit of 7° (Graph 4a). The prototype marking device-with-freehand resection was 0.8° better on average than the prototype marking device-with-sawguide and 1.6° better on average than the conventional-sawguide. All three resection techniques left the patella thicker superiorly on average, although the marking device results averaged closer to zero. Surprisingly Geometry 1 was worse than Geometry 2, and the difference was substantial, being 3.5° farther from the desired SI plane (p<0.001).

In the cadaveric testing, the SI angle was significantly affected by the interaction of experimenter and technique (p=0.01): Experimenter 2 achieved significant and dramatically better results with the marking device, within 1.6° of the symmetric plane with the marking device compared to 10.1° with the conventional sawguide (p=0.02) (Graph 4b). The lateral approach had the worst symmetry for the conventional sawguide, but was similar to the medial approaches for the marking device resections. The predicted peg results and the symmetric anterior surface were within a few degrees of each other (1.5° on average), confirming that the pegs predicted a symmetric resection.

### Bone Remnant Thickness

In the artificial bone testing, Geometry 1 (smaller and more regular) was significantly worse, by 1 mm, than Geometry 2 for obtaining the desired remaining bone thickness (p<0.001) (Graph 5a). The average thickness for Geometry 2 was very close to the desired thickness. The maximum thickness difference was 2.1 mm thinner than intended. The prototype marking device-with-sawguide had the widest range of thicknesses whereas the conventional-sawguide technique had the smallest range. All three techniques tended to leave the patella thinner than desired.

In the cadaveric testing, the remaining bone thickness was significantly affected by technique (p<0.001) (Graph 5b). The conventional-sawguide technique left the patellae in all cases over-resected, by an average of 0.9 mm. The prototype left all but one patella under-resected with the average being 1 mm thicker than desired. The worst thickness in the conventional case was for the lateral approach, whereas the prototype thickness for the lateral approach was similar to those for the medial approach.

### Procedure Time

In the artificial bone testing, the time taken for the procedure was significantly affected by experimenter (p=0.002) and geometry (p=0.04) (Graph 6a). Experimenter 2 took 50 seconds longer on average to complete a resection. The prototype marking device-with-freehand and the prototype marking device-with-sawguide were 28 seconds and 12 seconds faster on average than the conventional-sawguide, respectively. Geometry 1 was done 35 seconds faster on average than Geometry 2. While overall time for all 3 resection techniques was similar, the breakdown between the times was different (Graph 7a) since the experimenters were allowed to perform re-cuts with the conventional sawguide method but not with the two prototype techniques, whereas the prototype had more initial time leading up to the resection. The main time consumption and outliers related to securing the patella with the sawguide or towel clips, and performing the cut.

In the cadaveric testing, resection time was significantly affected by experimenter (p=0.03) (Graph 6b): Experimenter 1 completed the resections 1.2 minutes quicker on average than Experimenter 2. The prototype marking device-with-sawguide resection took 1.9 minutes less time than conventional-sawguide resection on average. As with the artificial bone testing, the prototype took more time initially whereas the conventional took more time later (Graph 7b).

Reactions from Evaluators

The experimenters considered the device a useful learning tool to create better resections, especially for less experienced surgeons. They appreciated the guidance that it provided, so that they did not need to struggle to decide where to put the sawguide, plus the fact that the resident or surgeon can confirm that they are satisfied with the line before proceeding with the cut. The expert surgeons we interviewed like that it provides guidance without constraining them to a particular resection line, and allows them to continue using the freehand or sawguide technique that they are already familiar with.

### DISCUSSION

The prototype marking device demonstrated symmetries that were equivalent or better on average than the conventional technique in a similar amount of time. Multiple cuts were permitted with the conventional technique whereas only a single cut was permitted with the prototype resections. The large range of angles for the conventional-sawguide resections shows that the normal procedure of caliper measurements and visual judgement is insufficient to produce symmetric cuts, especially in the SI direction. Further improvements in the prototype design, based on this pilot testing, together with more experience using the device, should lead to additional increases in accuracy and reductions in time; this is necessary before clinical implementation. Coupled with the positive qualitative feedback from the experimenters, this device offers the potential to improve training and confidence and reduce postoperative complications.

The more accurate ML angle with Geometry 1 was expected since it was chosen to be the easier patella to resect. Geometry 2 was larger and more complex and the experimenters had trouble securing the sawguide to it, causing the sawguide to shift. Since the same peg contact points were used in a post-resection checking device, which correctly identified the resulting asymmetries, the execution of the cut can be identified as the source of the error and not the prototype device itself. In the conventional-sawguide case, since subsequent cuts were made freehand, the sawguide shift did not present a problem. Experimenter 1 set the depth such that the cautery line was removed with the saw, as originally intended; however, the line was then no longer available as a check if the sawguide slipped. We therefore recommend setting the depth greater by 1 mm, or building this into the device. The outlier ML resection was likely due to the lateral approach, which the experimenters said threw them off (since the truncated knee specimen can easily be turned one way or the other, directions were less intuitive); the bone was also very hard and sclerotic, and the saw had trouble getting through the bone.

The source of SI inaccuracies in the artificial bone testing is likely because this patellar geometry was the greatest outlier (by 4°) when determining the peg configuration, but was still chosen since it had the least-consistently drawn angles by the surgeons, indicating the most uncertainty regarding the desired resection. The resection results were similar to this 4° offset while remaining within the symmetry limit of 7°; all other patellae examined had a smaller offset. In the cadaveric testing, Experimenter 2 appears to have mounted the device higher than the centre, which caused the device to have an angle relative to the anterior surface. We recommend incorporating a viewhole that can be placed over the centremark to ensure that the desired centring is achieved. It is also contemplated that a further device or procedure to aid with the centering could also be used. Experimenter 2 took on average 30 seconds longer than Experimenter 1 to ensure that the device was centred.

In the artificial bone testing, patellar geometry affected thickness for the conventional-sawguide but not the prototype resections. The prototype device can be used to resect at the final depth directly, or can be used first with a conservative thickness to guide the angle, followed by a final cut after checking the thickness. This could be particularly valuable if the patella is not everted.

Experimenter 2 took longer to perform the resections than Experimenter 1, but achieved dramatically better results with the prototype than conventional in the SI cadaveric results, showing the benefit of the prototype device for less experienced surgeons. The prototype device could have a significant time advantage over conventional if the surgeon normally does multiple passes, or spends considerable time evaluating the landmarks to determine the correct resection plane.

The main limitation of this study was the small number of experimenters and specimens, reflecting the time required to perform the testing as well as the availability of specimens. Despite this, important similarities and differences in the prototype device and conventional results were revealed.

For clinical use, there should be fewer parts, which can be addressed through higher-volume manufacturing methods, and the plastic bearing should be replaced by metal for ease of sterilization. While the thumb and forefingers provide a good way of holding the device on the patella, it is possible to hold it in this position mechanically: a pointed connection or small contact point similar to a towel clip can help hold it in place to release the fingers for applying the cautery mark; a clamp with a large contact area should be avoided as this tends to tilt the contact points off the anterior surface or tilt the patella.

### Example 3

### Measuring and Evaluating Tissue Resection

### Evaluation Device

The objective of this study was to evaluate the accuracy and usability of a prototype resection evaluation device designed to visually check the three-dimensional symmetry and thickness of the patellar bone remnant.

The thickness measurement is numeric whereas the angle evaluation is visual. Making the angle judgement a visual check rather than using exact numbers provides yes/no (symmetric/asymmetric) guidance regarding a recut, while keeping the device simple, quick and robust. Green light/red light guidance is as much as most surgeons desire, since the threshold for asymmetry is 7°. Quantitative feedback is possible by incorporating marker arrays for use with computer-assisted surgery (CAS).

### Methods:

### Device Design

The prototype evaluation device consists of three main components: (1) a three-peg contact with the anterior surface; (2) a swivel-plate contact with the resection surface (similar to an F-clamp); and (3) a sliding mechanism that measures the distance between the two surfaces.

As discussed above, a patellar referencing system consisting of three pegs in contact with the anterior surface was used. The pegs form a 16-mm equilateral triangle, with two superior points and one inferior, centred on the patella. The key concept behind the prototype device is that the frame adjacent to the swivel-plate is parallel to the anterior surface; therefore, the angle between the swivel plate and the frame shows visually the ML and SI angles between the resection and anterior surfaces. Once the device is set in place, the dovetail sliding mechanism is secured with a set-screw, and the thickness of the bone remnant read off the scale.

The prototype device includes extra features that make it suitable for use with a CAS system, such as divots to digitize the planes of the two plates, and support for the marker array; these could be removed in a revised, stand-alone version.

The anterior surface plate includes a viewhole to locate the device over the centrepoint of the patella, marked with a marker pen or cautery tool to make it quick and repeatable to position on the patella.

A small screw on the underside of the plate, beside the pegs, allows the plate to be flipped to suit whether it is a right or left patella and medial or lateral incision. The arrow points superiorly and the dovetail slider is on the incision side of the patella.

### Artificial Bone Testing

The prototype evaluation device was applied to 36 resected custom-moulded patellae, which included two different geometries, based on the most and least consistently identified resection planes from the patellae analyzed by the four surgeons. The patellae were resected by two senior Orthopaedic Surgery residents using freehand and sawguide techniques, resulting in patellae with ML angles ranging from +5° (medial side thicker/lateral side thinner) to −3° and SI angles ranging from +8° (superior side thicker/inferior side thinner) to −3°. Thicknesses varied from 2.1 mm over-resected to 1.3 mm under-resected. A third observer evaluated the results. Observations were noted regarding the interpretation of symmetry and the measured thickness, within 0.5 mm.

Following the observations, the patellae were imaged using computed tomography (CT), with slice thicknesses of 0.6 mm, segmented in Amira (Version 5.3.1; Visage Imaging, Andover, Mass.) and imported into AutoCAD (Version 2010, AutoDesk, San Rafael, Calif.) for measurement. The observations were compared to a quantitative analysis of the symmetry and thickness in which the resulting resection plane was compared to the average desired resection plane identified by the four experienced surgeons on these specific patellae. The desired thickness was established prior to testing based on restoring the original patellar thickness once the patellar component was added. A stringent threshold was set in that “good” was considered to have an angle<1°; “slightly thicker” was considered to have an angle between 1 and 2.2° and “thicker” referred to an angle>2.3°. Observations deviating from these ranges were highlighted.

### Cadaveric Testing

Eight pairs of fresh-frozen cadaveric knee specimens (6 female, 2 male; mean age 82, range 67 to 90 years) were used to validate the prototype evaluation device, following IRB approval. The resections were performed by the same two Orthopaedic Surgery residents using a sawguide technique. They observed and commented on the symmetry results using the evaluation device, and indicated whether or not they would recut the patella on the basis of what they saw. Experimenter 2 also reported the thickness as measured by the evaluation device and the thickness using standard patellar calipers (this was not done with Experimenter 1 because our initial focus was on the angles and resection accuracy). The time required to apply the device was recorded, as well as any overall comments from the experimenters.

Before and after testing, the specimens were CT scanned (0.6 mm slice thickness), segmented in Amira and models generated in AutoCAD. The symmetric resection plane was determined by fitting a plane to the central portion of the anterior surface, 15 mm in from the edge.

### Results:

In the artificial bone testing, the evaluation device accurately estimated symmetry in all cases for the ML angle and in 31/36 cases for the SI direction (Table 2). The more asymmetric patellae were all identified as such by the observer. Of the cases that were not estimated correctly, three related to resection angles less than 2.3°, which is much less than the asymmetry threshold of 7°. The fourth discrepancy (4.6° resection) was due to a lip remaining on the bone surface that lifted the plate off of the main resection surface.

The difference in thickness between the evaluation device reading and the CT bone measurement averaged −0.6 mm (SD 0.7 mm); the evaluation device indicated that the patella was slightly thinner on average than the CT measurement (Table 2).

In the cadaveric testing, the observations matched the quantitative measurements in 22/32 cases (Table 3). In the three ML cases where there was a discrepancy, the observer indicated that it was slightly thicker laterally when in fact the true angle was less than 1°. The seven SI discrepancies are discussed in greater detail below.

The difference in thickness between the prototype evaluation device reading and the CT bone measurement averaged+0.1 mm (SD 0.7 mm); in other words, the average evaluation device reading was very similar to the CT measurement (Table 3). By comparison, the caliper measurements averaged 1.6 mm (SD 0.6 mm) different than actual, including the thickness of the tissue overlying the patella.

What is claimed is:

1. A method for resecting tissue, said method comprising:
positioning a marking device into a desired position relative to a surface of a bone;
coupling the marking device to the surface of the bone in the desired position;
actuating the marking device thereby marking the bone to indicate a desired resection plane;
uncoupling the marking device from the surface of the bone; and
resecting a portion of the bone adjacent the marking.

2. The method of claim 1, wherein the desired position comprises a contact plane defined by contact between the marking device and an anterior surface of a patella, and wherein the contact plane defines a resection plane.

3. The method of claim 2, wherein the resection plane is substantially parallel with the contact plane.

4. The method of claim 1, wherein positioning the marking device comprises aligning the marking device with a reference point on the surface of the bone.

5. The method of claim 4, wherein the reference point comprises a center point of a patella, and wherein the center point is a midpoint between superior and inferior extents of the patella, and wherein the center point is a midpoint between medial and lateral extents of the patella.

6. The method of claim 1, wherein coupling the marking device comprises holding the device against the bone with a surgeon's hand.

7. The method of claim 1, wherein coupling the marking device comprises holding the device against the bone with a clamp.

8. The method of claim 1, wherein coupling the marking device comprises engaging an anchoring element against the bone, the anchoring element comprising a plurality of pegs.

9. The method of claim 8, wherein the plurality of pegs comprises three pegs arranged in a triangle.

10. The method of claim 9, wherein the triangle is an equilateral triangle.

11. The method of claim 10, wherein the equilateral triangle has sides having a length of about 16 mm each.

12. The method of claim 9, wherein two of the pegs in the triangle are oriented superiorly and one of the pegs in the triangle is oriented inferiorly.

13. The method of claim 1, wherein the bone comprises a patella, and wherein the resection plane is substantially parallel with an anterior portion of the patella.

14. The method of claim 1, wherein the bone comprises a patella, and wherein the resection plane is disposed at a fixed angle relative to an anterior surface of the patella.

15. The method of claim 1, wherein actuating the marking device comprises rotating the marking device relative to the surface of the bone.

16. The method of claim 15, wherein the marking device is coupled to the bone along an axis, and wherein the marking device is rotated in a plane substantially orthogonal thereto.

17. The method of claim 1, wherein marking the bone comprises marking a line on the bone.

18. The method of claim 17, wherein marking the line comprises marking the line at least 180 degrees around the bone.

19. The method of claim 17, wherein marking the bone comprises marking the bone with an electrosurgical device or a marker pen.

20. The method of claim 1, wherein uncoupling the marking device comprises disengaging an anchoring element from the bone.

21. The method of claim 1, wherein resecting the bone comprises sawing or reaming the bone adjacent the marking.

22. The method of claim 1, wherein the resection plane is generally parallel to the surface of the bone.

23. The method of claim 1, wherein the bone comprises a patella, and wherein the surface comprises an anterior surface of the patella.

24. The method of claim 1, wherein the marking device comprises an anchor for anchoring the device and a marking component for marking the bone, the method further comprising adjusting a distance between the anchor and the marking component.

25. The method of claim 1, further comprising orienting the device relative to the bone so that a portion of the device faces superiorly.

26. The method of claim 1, further comprising:
measuring natural thickness of the bone prior to resection thereof; and
adjusting the marking device to mark the bone by subtracting thickness of a desired amount of bone to remove from the natural thickness.

27. A marking device for use in tissue resection, said device comprising:
an elongate central member;
an anchoring arm having a first end and a second end opposite the first end, wherein the second end is coupled with the central elongate member;
an anchoring element coupled to the first end of the anchoring arm; and
a marking arm coupled to the central elongate member;
wherein the marking arm comprises a marking instrument coupled thereto, the marking instrument adapted to mark tissue, and wherein the marking arm moves relative to the anchoring element.

28. The device of claim 27, wherein the elongate central member comprises indicia for measuring distance.

29. The device of claim 27, further comprising a locking mechanism coupled with the elongate central member and the anchoring arm, the locking mechanism adapted to prevent movement therebetween.

30. The device of claim 29, wherein the locking mechanism comprises a setscrew.

31. The device of claim 27, wherein the anchoring arm is adjustably movable relative to the elongate central member.

32. The device of claim 31, wherein the anchoring arm slidably engages the elongate central member.

33. The device of claim 27, wherein the anchoring element comprises a base having a plurality of pegs disposed therein, the pegs adapted to anchor the device against the tissue.

34. The device of claim 33, wherein the anchoring arm is rotatably coupled with the base.

35. The device of claim 33, wherein the plurality of pegs comprise three pegs arranged in a triangle.

36. The device of claim 35, wherein the triangle is an equilateral triangle.

37. The device of claim 36, wherein the equilateral triangle comprises sides having a length of about 16 mm.

38. The device of claim 35, wherein two of the pegs in the triangle are oriented superiorly and one of the pegs in the triangle is oriented inferiorly.

39. The device of claim 33, wherein the base comprises indicia for indicating a superior or inferior orientation of the device relative to a patient.

40. The device of claim 33, wherein the pegs comprise conical distal tips adapted to engage bone.

41. The device of claim 27, wherein the anchoring element comprises a through hole disposed therein, the through hole adapted to allow visualization of a target therethrough.

42. The device of claim 41, further comprising a crosshair disposed in the through hole, the crosshair adapted to facilitate centering of the device over the target.

43. The device of claim 27, wherein the marking arm comprises a collar for engaging the marking instrument.

44. The device of claim 27, wherein the marking instrument comprises an electrosurgical instrument or a marking pen.

45. The device of claim 27, wherein the marking arm is movable relative to the anchoring arm.

46. The device of claim 27, wherein the tissue to be resected comprises bone, and the anchoring element anchors to the bone along an axis, and wherein the marking arm rotates in a plane substantially orthogonal to the axis.

47. The device of claim 27, wherein the marking arm has a longitudinal axis, and the marking instrument rotates in a plane substantially orthogonal to the longitudinal axis of the marking arm.

48. A method for evaluating tissue after resection, said method comprising:
positioning a measuring device into a desired position relative to a first surface of a bone after resection thereof, wherein the measuring device comprises a first base and a second base;
coupling the first base to the first surface of the bone in the desired position;
engaging the second base with a resected surface of the bone;
measuring thickness of the bone; and
determining an angle of the resected surface relative to the first surface of the bone.

49. The method of claim 48, wherein the desired position comprises a center of the bone.

50. The method of claim 49, wherein the bone comprises a patella.

51. The method of claim 49, wherein the center of the bone comprises a midpoint of medial and lateral extents of the bone, and the center of the bone comprises a midpoint of superior and inferior extents of the bone.

52. The method of claim 48, wherein the first base comprises a plurality of pegs, and wherein coupling the first base to the first surface comprises engaging the plurality of pegs with the first surface, the plurality of pegs disposed around the center of the bone.

53. The method of claim 48, wherein coupling the first base comprises observing a marked portion of the bone through an alignment hole disposed in the first base.

54. The method of claim 53, wherein observing the marked portion comprises aligning the marked portion of the bone with a crosshair disposed in the alignment hole.

55. The method of claim 48, wherein engaging the second base with the resected surface comprises advancing the second base toward the first base until a surface of the second base abuts the resected surface.

56. The method of claim 55, wherein the second base is substantially flush with the resected surface.

57. The method of claim 48, wherein measuring the thickness comprises reading a scale disposed on the measuring device, wherein the scale indicates thickness of the bone remnant, and wherein the scale is proportional to the distance between the two bases.

58. The method of claim 48, wherein the angle comprises a superoinferior angle or mediolateral angle of the resected surface relative to the first surface.

59. The method of claim 48, wherein the measuring device comprises a frame and the second base is pivotably coupled thereto, and wherein measuring the angle comprises observing an angle between a surface of the base and the frame.

60. The method of claim 48, wherein measuring the angle comprises measuring a first angle and then measuring a second angle different than the first angle.

61. The method of claim 48, wherein the bone comprises a patella, and wherein the first surface comprises an anterior surface of the patella.

62. The method of claim 48, further comprising conducting additional resection of the bone based on the measured thickness or angle.

63. The method of claim 48, wherein the measuring device comprises a marker array adapted for use with a computer-assisted surgery system, and wherein measuring the thickness or angle comprises:
detecting position and orientation of the marker array with the computer-assisted surgery system; and
digitizing divot positions on the measuring device.

64. A device for evaluating tissue after resection, said device comprising:
an elongate central member;
an anchoring arm having a first end and a second end opposite the first end, wherein the second end is coupled with the central elongate member;
an anchoring element coupled to the first end of the anchoring arm, the anchoring element adapted to engage a first surface of a bone;
a measuring arm having a first end and a second end opposite the first end, wherein the second end of the measuring arm is coupled with the central elongate member; and
a swivel base pivotably coupled to the first end of the measuring arm, wherein the swivel base is movable relative to the anchoring element, and wherein the swivel base is adapted to engage a resected surface of the bone substantially flush thereto.

65. The device of claim 64, wherein the elongate central member comprises indicia for measuring thickness of the remaining bone, wherein the thickness measured is proportional to distance between the anchoring element and the swivel base.

66. The device of claim 64, wherein the anchoring arm is adjustably movable relative to the elongate central member.

67. The device of claim 64, wherein the anchoring arm slidably engages the elongate central member.

68. The device of claim 64, wherein the anchoring element comprises a base having a plurality of pegs disposed therein, the pegs adapted to anchor the device against the tissue.

69. The device of claim 68, wherein the plurality of pegs comprise three pegs arranged in an equilateral triangle.

70. The device of claim 69, wherein the equilateral triangle comprises sides having a length of about 16 mm.

71. The device of claim 70, wherein two pegs in the equilateral triangle are oriented superiorly and one of the pegs in the equilateral triangle is oriented inferiorly.

72. The device of claim 68, wherein the plurality of pegs comprise conical distal tips adapted to engage bone.

73. The device of claim 64, wherein the anchoring element comprises a base, and wherein the base comprises indicia for indicating a superior or inferior orientation of the device relative to a patient.

74. The device of claim 64, wherein the anchoring element comprises a through hole disposed therein, the through hole adapted to allow visualization of a target therethrough.

75. The device of claim 74, further comprising a crosshair disposed in the through hole, the crosshair adapted to facilitate centering of the device over the target.

76. The device of claim 64, wherein the measuring arm is movable relative to the anchoring arm.

77. The device of claim 64, wherein the measuring arm is slidably engaged with the elongate central member.

78. The device of claim 64, wherein the swivel base is pivotably coupled to the measuring arm with a ball joint.

79. The device of claim 64, wherein a gap between the swivel base and the measuring arm provides a visual indicator of a superoinferior angle or a mediolateral angle of the resected surface of bone relative to the first surface.

80. The device of claim 64, further comprising a scale operably coupled with the measuring arm, wherein the scale indicates a superoinferior angle or a mediolateral angle of the resected surface of the bone relative to the first surface.

81. A computer assisted surgical system, said system comprising:
one or more marker arrays coupled with the device of claim 64; and
a computer assisted surgical system, wherein the computer assisted surgical system is adapted to detect the one or more marker arrays and determine angle of the resected surface and depth of the bone remnant.

82. The system of claim 81, wherein the computer assisted surgical system optically detects the one or more marker arrays.

83. A method for training a surgeon to perform patella resection, said method comprising:
coupling a marking device to a patella, wherein the patella comprises a patient's patella or a model of a patella;
marking the patella with the marking device to indicate a region for resection;
removing the marking device from the patella; and
comparing an intended resection plane before resecting the patella with the marked region or comparing the patella after resection with the marked region.

84. The method of claim 83, further comprising coupling a measuring instrument to the resected patella and determining thickness thereof and/or a resection angle of the resected surface relative to an anterior surface of the patella.
